(19) United States
(12) Patent Application Publication
(10) Pub. No.: US 2025/0000802 A1
(43) Pub. Date: Jan. 2, 2025

(54) SOLID FORMULATIONS COMPRISING AN INHIBITOR OF THE K-RAS PROTEIN HAVING A G12C MUTATION, AND A PROCESS FOR PREPARING

(71) Applicants: HOFFMANN-LA ROCHE INC., Little Falls, NJ (US); GENENTECH, INC., South San Francisco, CA (US)

(72) Inventors: Samuel Hsiao-Chieh YANG, San Francisco, CA (US); Po-Chang CHIANG, South San Francisco, CA (US); Chen MAO, Foster City, CA (US); Avinash MURTHY, Basel (CH); Claudia VICENTE MARTIN, Basel (CH)

(73) Assignees: HOFFMANN-LA ROCHE INC., Little Falls, NJ (US); GENENTECH, INC., South San Francisco, CA (US)

(21) Appl. No.: 18/734,329

(22) Filed: Jun. 5, 2024

Related U.S. Application Data
(60) Provisional application No. 63/507,245, filed on Jun. 9, 2023.

(51) Int. Cl. A61K 9/20; A61K 31/517

(52) U.S. Cl. CPC A61K 9/2054; A61K 9/2009; A61K 9/2095; A61K 31/517

(57) ABSTRACT

The present disclosure relates generally to rapid-release pharmaceutical dosage unit tablets containing small molecule API medicines. In some instances the API is an inhibitor of Ras proteins, such as K-Ras, H-Ras, and N-Ras, that have a G12C mutation. Such tablets also contain a disintegrant and an excipient. More specifically, the present disclosure relates to pharmaceutical dosage unit tablets containing divarasib, or a pharmaceutically acceptable salt thereof, an extragranular disintegrant, and an extragranular excipient, and to processes for preparing the tablets from granules formed by dry granulation.

### BACKGROUND OF THE DISCLOSURE

This application claims benefit of priority to U.S. Provisional Application No. 63/507,245 filed Jun. 9, 2023, which is incorporated herein by reference in its entirety.

The present disclosure relates generally to pharmaceutical dosage unit tablets containing a drug, a disintegrant, and an excipient, and processes for forming the tablets.

In some aspects of the disclosure, the drug is an inhibitor of Ras proteins. Ras is a small GTP-binding protein that functions as a nucleotide-dependent switch for central growth signaling pathways. In response to extracellular signals, Ras is converted from a GDP-bound (RasGDP) to a GTP-bound (RasGTP) state, as catalyzed by guanine nucleotide exchange factors (GEFs), notably the SOS1 protein. Active RasGTP mediates its diverse growth-stimulating functions through its direct interactions with effectors including Raf, PI3K, and RaI guanine nucleotide dissociation stimulator. The intrinsic GTPase activity of Ras then hydrolyzes GTP to GDP to terminate Ras signaling. The Ras GTPase activity can be further accelerated by its interactions with GTPase-activating proteins (GAPs), including the neurofibromin 1 tumor suppressor.

Mutant Ras has a reduced GTPase activity, which prolongs its activated conformation, thereby promoting Ras-dependent signaling and cancer cell survival or growth. Mutation in Ras which affects its ability to interact with GAP or to convert GTP back to GDP will result in a prolonged activation of the protein and consequently a prolonged signal to the cell telling it to continue to grow and divide. Because these signals result in cell growth and division, overactive RAS signaling may ultimately lead to cancer. Mutations in any one of the three main isoforms of RAS (H-Ras, N-Ras, or K-Ras) genes are common events in human tumorigenesis. Among the three Ras isoforms (K, N, and H), K-Ras is most frequently mutated.

The most common K-Ras mutations are found at residue G12 and G13 in the P-loop and at residue Q61. G12C is a frequent mutation of K-Ras gene (glycine-12 to cysteine). G12D and G13D are other frequent mutations. Mutations of Ras in cancer are associated with poor prognosis. Inactivation of oncogenic Ras in mice results in tumor shrinkage. Thus, Ras is widely considered an oncology target of exceptional importance.

Immediate-release compressed tablets (e.g., caplets) are an effective form for delivering pharmaceutically active drugs. Problematically, some combinations of drugs, excipients, and disintegration agents (to impart rapid release properties) result in tablets that have slow disintegration times. Early formulations comprising divarasib had slow disintegration times and incomplete disintegration of the tablet after long times in the stomach. Quick delivery of the divarasib API is necessary to avoid changes in, for example, pharmacokinetic properties. A need therefore exists for improved tablet compositions containing a drug (e.g. divarasib), an excipient, and a disintegration agent and related preparation processes that allow for tablets having adequate dissolution and disintegration characteristics required for immediate-release tablets.

### BRIEF DESCRIPTION OF THE DISCLOSURE

In some aspects of the disclosure, a process for preparing a pharmaceutical dosage unit tablet core is provided. The process comprises:
(a) blending divarasib, an intragranular excipient, and an intragranular disintegrant to form a pre-blend;
(b) compacting the pre-blend by application of a compaction force;
(c) milling and screening the compacted pre-blend to form granules;
(d) blending the granules with an extragranular excipient and an extragranular disintegrant to form a granule blend; and
(e) tableting the granule blend by application of a tablet compaction force to form the pharmaceutical dosage unit tablet core.

In one aspect, the process further comprises, prior to compaction, blending the pre-blend with an intragranular lubricant. In another aspect, the process further comprises, prior to tableting, blending the granules, the extragranular excipient, and the extragranular disintegrant with an extragranular lubricant to form the granule blend.

In other aspects of the disclosure, a pharmaceutical dosage unit tablet core is provided. The tablet core comprises:
(a) divarasib;
(b) a disintegrant at a content based on the pharmaceutical dosage unit tablet core weight of from about 2 wt % to about 13 wt %; and
(c) an excipient at a content based on the pharmaceutical dosage unit tablet core weight of from about 15 wt % to about 50 wt %.

### BRIEF DESCRIPTION OF THE DRAWINGS

FIG. 1 depicts a tableting process of the present disclosure.

FIG. 2 shows the effect of various excipients on disintegration time, as discussed in Example 2. MCC=microcrystalline cellulose; DCP=dicalcium phosphate.

FIG. 3 shows the effect of extragranular microcrystalline cellulose content on average disintegration time, as discussed in Example 3. MCC=microcrystalline cellulose; ExG=extragranular; InG=intragranular; TS=tensile strength; NaCMC=croscarmellose sodium.

FIG. 4 shows the effect of intragranular and extragranular croscarmellose sodium on average disintegration time, as discussed in Example 4. ExG=extragranular; InG=intragranular; TS=tensile strength; NaCMC=croscarmellose sodium.

FIG. 5 shows the effect of extragranular microcrystalline cellulose grade on average disintegration time, as discussed in Example 5. MCC=microcrystalline cellulose; ExG=extragranular; InG=intragranular; TS=tensile strength; NaCMC=croscarmellose sodium.

FIG. 6 shows the disintegration behavior of tablets formulated by dry granulation in comparison to tablets formulated by high shear wet granulation, as discussed in Example 6. MCC=microcrystalline cellulose; ExG=extragranular; InG=intragranular; TS=tensile strength; NaCMC=croscarmellose sodium; DG=dry granulation; HSWG=high shear wet granulation; SF=solid fraction.

FIG. 7 is a flow chart illustrating the manufacturing process and in-process characterization of divarasib tablets, as discussed in Example 7. API=active pharmaceutical ingredient (i.e., divarasib).

FIG. 8 shows the tabletability profile of divarasib tablets, as discussed in Example 7.

FIG. 9 shows the disintegration time of tablets with a target hardness of 20 and 30 kp, as discussed in Example 7. DL=drug load; CCS=croscarmellose sodium; Ave=average.

### DETAILED DESCRIPTION OF THE DISCLOSURE

In accordance with some aspects of the present disclosure, there is provided, as described herein, pharmaceutical dosage form tablets comprising up to about 44 weight percent (“wt. %”) free base (FB) content of an active drug, a disintegrant, and an excipient, wherein the tablets exhibit rapid dissolution rate. In accordance with some other aspects of the present disclosure, there is provided, as described herein, processes for preparing the pharmaceutical dosage form tablets comprising an active drug from granules.

In some aspects of the present disclosure the active drug is 1-((S)-4-((R)-7-(6-amino-4-methyl-3-(trifluoromethyl)pyridin-2-yl)-6-chloro-8-fluoro-2-(((S)-1-methylpyrrolidin-2-yl)methoxy)quinazolin-4-yl)-3-methylpiperazin-1-yl)prop-2-en-1-one freebase or a pharmaceutically acceptable salt thereof (hereinafter referred to as “divarasib”). Divarasib has the following structure:

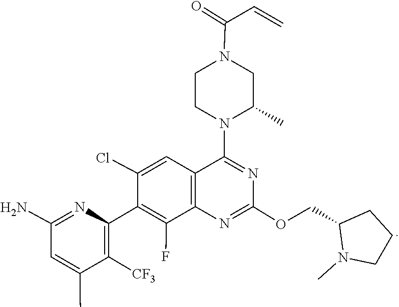

The term “divarasib freebase” as used herein refers to divarasib (1-((S)-4-((R)-7-(6-amino-4-methyl-3-(trifluoromethyl)pyridin-2-yl)-6-chloro-8-fluoro-2-(((S)-1-methylpyrrolidin-2-yl)methoxy)quinazolin-4-yl)-3-methylpiperazin-1-yl)prop-2-en-1-one) as a freebase (i.e. not as a salt).

Divarasib, preparative methods, and therapeutic uses thereof are disclosed in International Publication Number WO 2020/097537, which is incorporated herein by reference. In one aspect, divarasib is prepared according to the procedure set forth in Example 17a of WO 2020/097537, which is herein incorporated by reference.

The term “divarasib adipate” as used herein refers to the adipate salt form of divarasib (1-((S)-4-((R)-7-(6-amino-4-methyl-3-(trifluoromethyl)pyridin-2-yl)-6-chloro-8-fluoro-2-(((S)-1-methylpyrrolidin-2-yl)methoxy)quinazolin-4-yl)-3-methylpiperazin-1-yl)prop-2-en-1-one adipate).

In some aspects, divarasib adipate is prepared by dissolving divarasib freebase in a solvent (e.g., methylethylketone, “MEK”), dissolving adipic acid in a solvent (e.g., MEK), and combining the divarasib freebase solution with the adipic acid solution to form divarasib adipate. In certain embodiments, gentle heating (40-80° C.) may be necessary to ensure complete dissolution. In some embodiments, the process further comprises adding seed crystals of the divarasib adipate to the mixture. In one embodiment, divarasib adipate is synthesized as described, for example, in WO2022035790. In another embodiment, divarasib adipate is synthesized as described, for example, in WO2023/150653.

Special reference is made in the description to the active drug divarasib, however, the compositions, formulations and processes disclosed herein are applicable to and encompass active drugs other than divarasib.

It has been discovered that water penetration into divarasib tablets affects disintegration time of the tablets. As demonstrated in the examples herein, it has further been discovered that inclusion of certain amounts of extragranular (ExG) water-sorbing excipients, in addition to extragranular disintegrants, improves water penetration into the tablets and results in substantially faster disintegration times. In one particular aspect, the tablets of the present disclosure have a disintegration time of about 15 minutes or less, about 10 minutes or less, or about 5 minutes or less, including about 10 minutes, about 9 minutes, about 8 minutes, about 7 minutes, about 6 minutes, about 5 minutes, about 4 minutes, about 3 minutes, about 2 minutes, about 1.5 minutes, about 1.3 minutes, or about 1 minute, and ranges thereof, such as from about 1 minute to about 10 minutes, from about 1 minute to about 8 minutes, from about 1 minute to about 6 minutes, from about 1 minute to about 5 minutes, from about 1 minute to about 4 minutes, from about 1 minute to about 3 minutes, or from about 1 minute to about 2 minutes. Disintegration times may be measured using the process set forth in the examples.

As used herein, intragranular refers to a component that is added prior to granulation such that the component is incorporated within the granules. As further used herein, extragranular refers to a component that is combined with the granules prior to compression, such as in a tablet press.

### Tablet Components

Active Drug. The tablet compositions of the present disclosure comprise up to about 44 wt % or up to about 40 wt % of active drug (freebase content), including about 5 wt. % or more active drug, such as about 1 wt %, about 5 wt. %, about 10 wt. %, about 15 wt. %, about 20 wt. %, about 25 wt. %, about 30 wt. % about 35 wt. %, about 40 wt. %, or about 44 wt. % active drug (freebase content), and ranges thereof, such as from about 1 wt % to about 44 wt %, from about 1 wt % to about 40 wt %, from about 5 wt. % to about 44 wt. %, from about 10 wt. % to about 44 wt. %, from about 15 wt. % to about 44 wt. %, from about 20 wt. % to about 44 wt. %, from about 25 wt. % to about 44 wt. %, from about 30 wt. % to about 40 wt %, or from about 35 wt. % to about 40 wt. % of active drug (freebase content). As used herein, wt. % is given based on the pharmaceutical dosage unit tablet core weight, unless otherwise indicated.

The tablet compositions of the present disclosure may comprise up to about 60 wt %, or up to about 54 wt % of divarasib adipate, including about 5 wt. % or more divarasib adipate, such as about 1 wt %, about 5 wt. %, about 10 wt. %, about 15 wt. %, about 20 wt. %, about 25 wt. %, about 30 wt. % about 35 wt. %, about 40 wt. %, about 45 wt. %, about 50 wt. %, about 54 wt %, about 55 wt. %, or about 60 wt. % divarasib adipate, and ranges thereof, such as from about 1 wt % to about 60 wt %, from about 1 wt % to about 54 wt %, from about 5 wt. % to about 60 wt. %, from about 10 wt. % to about 60 wt. %, from about 15 wt. % to about 60 wt. %, from about 20 wt. % to about 60 wt. %, from about 25 wt. % to about 55 wt. %, from about 30 wt. % to about 50 wt %, or from about 35 wt. % to about 45 wt. % of divarasib adipate.

Excipient. The tablets of the present disclosure comprise one or more excipients. Excipients or fillers are conventionally included in tablets to assist with, for example, direct compression (tableting) of granules prepared by dry granulation. However, divarasib (e.g. divarasib adipate) tablets prepared using a conventional formulation approach have been found to have a slow tablet disintegration time (e.g., greater than 20 minutes). Without wishing to be bound to any particular theory, it is believed that water penetration into the tablet is a limiting factor in disintegration time of divarasib (e.g. divarasib adipate) tablets.

Based on experimental results to date, it has been discovered that inclusion of water-sorbing excipients in divarasib (e.g. divarasib adipate) tablets may improve the disintegration times of the tablets. In particular, it has been discovered that inclusion of certain amounts of extragranular (ExG) water-sorbing excipients result in substantially faster disintegration times for divarasib (e.g. divarasib adipate) tablets.

Excipient may suitably be added to the pre-blend prior to granulation thereby forming intragranular (InG) excipient. Additionally, excipient may be combined with granulated pre-blend prior to tableting thereby forming extragranular (ExG) excipient.

Thus, in one aspect, the tablets of the present disclosure comprise an excipient selected from the group consisting of microcrystalline cellulose (MCC), silicified MCC, pregelatinized starch, starch, and combinations thereof. In one embodiment, the excipient is selected from the group consisting of MCC and starch. In one particular embodiment, the excipient is MCC.

In certain aspects, the MCC may have an average particle size of from about 20 μm to about 180 μm, or from about 20 μm to about 100 μm, or from about 35 μm to about 95 μm, or from about 40 μm to about 75 μm, or from about 45 μm to about 55 μm, including about 20 μm, about 25 μm, about 30 μm, about 35 μm, about 40 μm, about 45 μm, about 50 μm, about 55 μm, about 50 μm, about 65 μm, about 70 μm, about 75 μm, about 80 μm, about 85 μm, about 90 μm, about 95 μm, or about 100 μm. In one particular embodiment, the MCC has an average particle size of about 50 um. Suitable MCC includes, but is not limited to Avicel® PH 101, Avicel® PH 102, and Avicel® PH 105. In one particular aspect, the MCC is Avicel® PH 101.

Total excipient content based on tablet core weight is about 15 wt. %, 16 wt. %, 17 wt. %, 18 wt. %, 19 wt. %, 20 wt. %, 21 wt. %, 22 wt. %, 23 wt. %, 24 wt. %, 25 wt. %, about 26 wt. %, about 27 wt. %, about 28 wt. %, about 29 wt. %, about 30 wt. %, about 31 wt. %, about 32 wt. %, about 33 wt. %, about 34 wt. %, about 35 wt. %, about 36 wt. %, about 37 wt. %, about 38 wt. %, or about 39 wt. %, about 40 wt. %, about 41 wt. %, about 42 wt. %, about 43 wt. %, about 44 wt. %, about 45 wt. %, about 46 wt. %, about 47 wt. %, about 48 wt. %, about 49 wt. %, or about 50 wt. %, and ranges thereof, such as from about 15 wt. % to about 50 wt. %, from about 20 wt. % to about 45 wt. %, from about 30 wt. % to about 40 wt. %, from about 32 wt. % to about 38 wt. %, from about 33 wt. % to about 37 wt. %, or from about 34 wt. % to about 36 wt. %.

Total intragranular (InG) excipient content based on tablet core weight is about 10 wt. %, about 11 wt. %, about 12 wt. %, about 13 wt. %, about 14 wt. %, about 15 wt. %, about 16 wt. %, about 17 wt. %, about 18 wt. %, about 19 wt. %, about 20 wt. %, about 21 wt. %, about 22 wt. %, about 23 wt. %, about 24 wt. %, about 25 wt. %, about 26 wt. %, about 27 wt. %, about 28 wt. %, about 29 wt. %, or about 30 wt. %, and ranges thereof, such as from about 10 wt. % to about 30 wt. %, from about 15 wt. % to about 25 wt. %, from about 16 wt. % to about 24 wt. %, from about 17 wt. % to about 23 wt. %, from about 18 wt. % to about 22 wt. %, from about 19 wt. % to about 21 wt. %, or from about 20 wt. % to about 21 wt. %.

As demonstrated in the examples herein, the amount of extragranular (ExG) excipient may affect the disintegration time of the divarasib (e.g. divarasib adipate) tablets. Specifically, use of certain water-sorbing excipients, such as MCC, surprisingly improves the water penetration into the tablet, and thus tablet disintegration time. Thus in one aspect, the total extragranular (ExG) excipient content based on tablet core weight is about 5 wt. %, about 6 wt. %, about 7 wt. %, about 8 wt. %, about 9 wt. %, about 10 wt. %, about 11 wt. %, about 12 wt. %, about 13 wt. %, about 14 wt. %, about 15 wt. %, about 16 wt. %, about 17 wt. %, about 18 wt. %, about 19 wt. %, or about 20 wt. %, and ranges thereof, such as from about 5 wt. % to about 20 wt. %, from about 8 wt. % to about 18 wt. %, from about 10 wt. % to about 20 wt. %, from about 10 wt. % to about 15 wt. %, from about 11 wt. % to about 19 wt. %, from about 12 wt. % to about 18 wt. %, or from about 14 wt. % to about 16 wt. %.

Disintegrant. Disintegrant is used to help ensure rapid disintegration of the tablet. This is achieved through, for example, rapid swelling when in contact with aqueous media. Disintegrant may suitably be added to the pre-blend prior to granulation thereby forming intragranular (InG) disintegrant. Additionally, disintegrant may be combined with granulated pre-blend prior to tableting thereby forming extragranular (ExG) disintegrant. Disintegrants are known in the art. Non-limiting examples include: modified starches such as sodium carboxymethyl starch (sodium starch glycolate, “SSG”); cross-linked polyvinylpyrrolidones such as crospovidone (“crossPVP”); modified celluloses such as croscarmellose sodium (“NaCMC” or “CCS”); cross-linked alginic acid; gums such as gellan gum and xanthan gum; calcium silicate. In some aspects of the disclosure, the disintegrant is croscarmellose sodium, crospovidone, sodium starch glycolate, or combinations thereof. In some aspects of the disclosure, the disintegrant is croscarmellose sodium.

Total tablet disintegrant loading based on tablet core weight may be about 2 wt. %, about 3 wt. %, about 4 wt. %, about 5 wt. %, about 6 wt. %, about 7 wt. %, about 8 wt. %, about 9 wt. %, about 10 wt. %, about 11 wt. %, about 12 wt. %, or about 13 wt. %, and ranges thereof, such as from about 2 wt. % to about 13 wt. %, from about 3 wt. % to about 12 wt. %, from about 4 wt. % to about 11 wt. %, from about 5 wt. % to about 10 wt. %, from about 6 wt. % to about 9 wt. % or from about 7 wt. % to about 8 wt. %.

Total tablet intragranular disintegrant loading based on tablet core weight may be about 1 wt. %, about 1.5 wt. %, about 2 wt. %, about 2.5 wt. %, about 3 wt. %, about 3.5 wt. %, about 4 wt. %, about 4.5 wt. %, or about 5 wt. %, and ranges thereof, such as from about 1 wt. % to about 5 wt. %, from about 1.5 wt. % to about 4.5 wt. %, from about 2 wt. % to about 4 wt. %, from about 2.5 wt. % to about 3.5 wt. %, or from about 2.75 wt. % to about 3.25 wt. %.

Total tablet extragranular disintegrant loading based on tablet core weight may be about 1 wt. %, about 1.5 wt. %, about 2 wt. %, about 2.5 wt. %, about 3 wt. %, about 3.5 wt. %, about 4 wt. %, about 4.5 wt. %, about 5 wt. %, about 5.5 wt. %, about 6 wt. %, about 6.5 wt. %, about 7 wt. %, about 7.5 wt. %, or about 8 wt. %, and ranges thereof, such as from about 1 wt. % to about 8 wt. %, from about 1.5 wt. % to about 7.5 wt. %, from about 2 wt. % to about 7 wt. %, from about 2.5 wt. % to about 6.5 wt. %, from about 3 wt. % to about 6 wt. %, from about 3.5 wt. % to about 5.5 wt. %, or from about 4 wt. % to about 5 wt. %.

Based on experimental results to date, surprisingly the total extragranular excipient and extragranular disintegrant level may affect water penetration into the tablet, and thus result in tablets having a shorter disintegration time. Thus, in one aspect, the total amount of extragranular excipient and extragranular disintegrant based on tablet core weight may be about 15 wt. %, about 16 wt. %, about 17 wt. %, about 18 wt. %, about 19 wt. %, about 20 wt. %, about 21 wt. %, about 22 wt. %, about 23 wt. %, about 24 wt. %, or about 25 wt. %, and ranges thereof, such as from about 15 wt. % to about 25 wt. %, from about 16 wt. % to about 24 wt. %, from about 17 wt. % to about 23 wt. %, from about 18 wt. % to about 22 wt. %, or from about 19 wt. % to about 20 wt. %.

Lubricant. Lubricants are added to compositions for tableting in order to reduce the friction between the surfaces of manufacturing equipment and that of organic solids in the tablet composition, promote ejection from the tablet press, affect the dynamics of dry granulation and tableting processes, and affect the mechanical properties of tablets. Lubricants may suitably be added to the pre-blend prior to granulation thereby forming intragranular lubricant. Additionally, lubricants may be combined with granulated pre-blend prior to tableting thereby forming extragranular lubricant. Lubricants are known in the art. Non-limiting examples include magnesium stearate, calcium stearate, stearic acid, sodium stearyl fumarate, hydrogenated vegetable oils, polyethylene glycol (4000-6000), and sodium lauryl sulfate. In some aspects of the disclosure, the lubricant is magnesium stearate, sodium stearyl fumarate, stearic acid, and combinations thereof. In one aspect, the lubricant is magnesium stearate.

Total tablet lubricant loading, on a tablet core basis, may be about 1 wt. %, about 1.25 wt. %, about 1.5 wt. %, about 1.75 wt. %, about 2 wt. %, about 2.25 wt. %, about 2.5 wt. %, about 2.75 wt. %, or about 3 wt. %, and ranges thereof, such as from about 1 wt. % to about 3 wt. %, from about 1.5 wt. % to about 2.5 wt. %, from about 1.75 wt. % to about 2.5 wt. %, or from about 2 wt. % to about 2.5 wt. %.

Total tablet intragranular lubricant loading, on a tablet core basis, may be about 0.1 wt. %, about 0.15 wt. %, about 0.2 wt. %, about 0.25 wt. %, about 0.3 wt. %, about 0.4 wt. %, about 0.5 wt. %, about 0.75 wt. %, about 1 wt. %, about 1.25 wt. %, about 1.5 wt. %, about 2.0 wt %, about 2.5 wt %, or about 3.0 wt %, and ranges thereof, such as from about 0.1 wt. % to about 1.5 wt. %, from about 0.2 wt. % to about 1.25 wt. %, from about 0.5 wt. % to about 1.25 wt. %, or from about 1 wt % to about 3 wt %.

Total tablet extragranular lubricant loading, on a tablet core basis, may be about 0.5 wt. %, about 0.75 wt. %, about 1 wt. %, about 1.25 wt. %, about 1.5 wt. %, about 1.75 wt. %, about 2 wt. %, about 2.25 wt. %, about 2.5 wt. %, or about 3 wt %, and ranges thereof, such as from about 0.5 wt. % to about 3 wt. %, from about 0.5 wt. % to about 2.5 wt. %, from about 0.75 wt. % to about 2.25 wt. %, from about 0.75 wt. % to about 2 wt. %, from about 1 wt % to about 3 wt %, from about 1 wt % to about 2 wt %, from about 1 wt. % to about 1.75 wt. % or from about 1.25 wt. % to about 1.5 wt. %. In some particular aspects, the lubricant is present intragranularly at levels ranging from 0.75% to 1.25% and extragranularly at levels ranging from 1% to 1.50% at a total loading 1.75% to 2.75%.

Tablet Coating. The tablets of the present disclosure (tablet cores) are preferably coated with a film-coating to provide for tablets that are predominantly tasteless and odorless, and are easy to swallow. Further, film coating prevents dust formation during packaging and ensures robustness during transportation. Commercial coating compositions are suitable for purposes of the present disclosure and include, but are not limited to, Opadry® YS grades, such as Opadry® YS-1-7003 and Opadry® YS-1-18202, and Opadry® II grades, such as Opadry® II White 85F18422, and Opadry® II Brown 85F26792. In any of the various coating aspects, the surface of the tablet core is coated with from about 2 wt. % to about 6 wt. %, or from about 2 wt. % to about 4 wt. % of a film-coating based on the weight of the table core. In some aspects of the disclosure the film coating comprises: from about 30 wt. % to about 50 wt. %, from about 35 wt. % to about 45 wt. % or from 38 wt. % to 42 wt. % of a coating agent (e.g., polyvinyl alcohol); from about 20 wt. % to about 30 wt. % or from 23.8 wt. % to 26.3 wt. % of a pigment (e.g., titanium dioxide); from about 15 wt. % to about 25 wt. % or from 19.2 wt. % to 21.2 wt. % of a plasticizer (e.g., Macrogol/PEG3350); and from about 10 wt. % to about 20 wt. % or from 14.1 wt. % to 15.5 wt. % of an anticaking agent (e.g., talc).

Optional Components. Other optional table core components include talc (an antiadherent/glidant), fumed silicon dioxide (i.e., colloidal silicon dioxide) (an antiadherent/glidant), citric acid (a pH adjuster) and tartaric acid (a pH adjuster).

### Tablet Manufacturing Process

The tableting manufacturing process of the present disclosure utilize standard and conventional pharmaceutical operations, such as screening, blending, dry granulation, compression, and film coating.

In one aspect, the process of the present disclosure comprises blending an active drug (e.g., divarasib adipate) with an intragranular excipient (e.g., MCC), and an intragranular disintegrant (e.g., NaCMC) to form a pre-blend, compacting the pre-blend by application of a compaction force; milling and screening the compacted pre-blend to form granules; blending the granules with an extragranular excipient (e.g., MCC) and an extragranular disintegrant (e.g., NaCMC) to form a granule blend; and tableting the granule blend by application of a tablet compaction force to form the tablet core. In certain aspects, the process may further comprise, prior to compaction, blending the pre-blend with an intragranular lubricant (e.g., magnesium stearate “MgSt”). In certain aspects, the process may further comprise, prior to tableting, blending the granules, the extragranular excipient, and the extragranular disintegrant with an extragranular lubricant to form the granule blend.

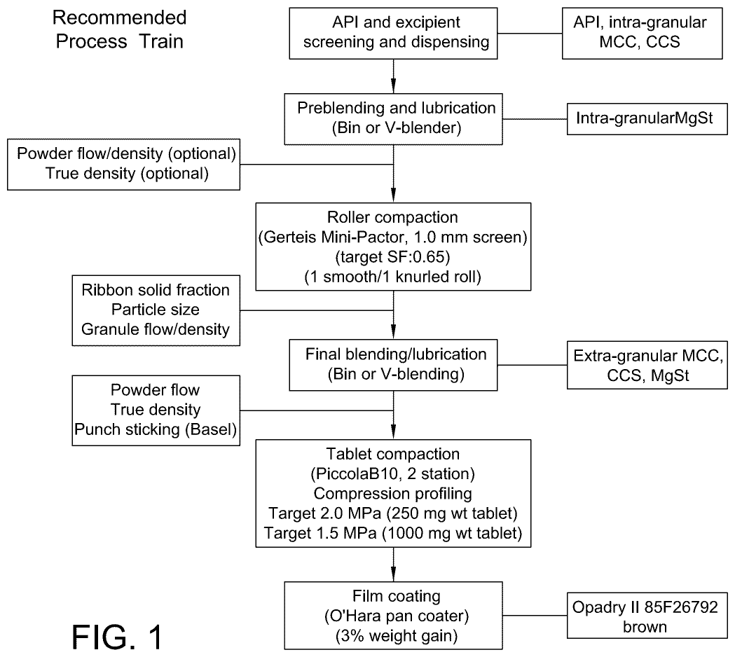

More specifically, in one process of the present disclosure depicted in FIG. 1, excipient and disintegrant are combined with an active drug (e.g. divarasib adipate) to form a pre-blend, and the pre-blend is screened. Lubricant is further combined with the pre-blend in a bin or blender. In some optional aspects, the excipient and disintegrant are screened, combined with the active drug (e.g. divarasib adipate) and blended for a first blend period followed by addition of screened lubricant and a blending in a second blend period to form the pre-blend. As noted herein, the excipient, disintegrant and lubricant in the pre-blend are termed intragranular. In one optional aspect, the powder flow, density, and/or true density of the pre-blend may be determined using conventional means.

The pre-blend is then processed by roller compaction followed by milling and screening to form granules. In one optional aspect, the particle size, granule flow, and density of the granules may be determined using conventional means. The granules are combined with additional screened lubricant, screened disintegrant, and screened excipient and blended to form a granule blend. In some optional aspects, the granules are combined with screened disintegrant and screened excipient and blended for a first period followed by addition of screened lubricant and blending in a second blend period to form the granule blend. The disintegrant, excipient, and lubricant combined with the granules are termed extragranular. In one optional aspect, the powder flow, true density, and punch sticking of the granule blend may be determined using conventional means. The granule blend is compressed using any suitable tableting apparatus known in the art to form tablet cores. The table cores are coated with a film coating to form the finished tablets.

In one particular process of the present disclosure, intragranular excipient (e.g., MCC) and disintegrant (e.g., croscarmellose sodium) are screened and combined with active drug (e.g., divarasib adipate) and admixed (blended) in a first pre-blending step. The first blend material is combined with screened lubricant (e.g., magnesium stearate) and admixed (blended) in a second pre-blending step to form the pre-blend. The pre blend is granulated by roller compaction, milled and screen to form granules. The granules are combined with screened extragranular excipient and screened extragranular disintegrant and admixed (blended) in a first final blending step. The first blend material is combined with screened extragranular lubricant and admixed (blended) in a second final blending step to form a granule blend. The granule blend is compressed using any suitable tableting apparatus known in the art to form tablet cores. Solid film coating material is combined with an aqueous carrier and suspended. The table cores are coated with the film coating suspension to form the finished tablets.

Pre-blendinq. Pre-blending is designed to provide substantial homogeneity of the intragranular components prior to roller compaction. Pre-blending equipment and related process parameters provide for essentially homogeneous blends are known to those skilled in the art and are not believed to be narrowly critical. Suitable blenders are known in the art and any apparatus typically employed in the pharmaceutical industry for uniformly admixing two or more components including V-shaped blenders, double-cone blenders, bin (container) blenders, and rotary drum blenders. The combination blender volume, blender fill, rotation speed and rotation time may be suitably determined by those skilled in the art, based on routine experimentation, to achieve an essentially homogeneous admixture of components. Blender volume is suitably 5L, 10L, 25L, 50 L, 100 L, 200 L, 250 L or greater. Selection of blender fill allows for convection and three-dimensional material movement and is suitably about 25%, about 30%, about 35%, about 40%, about 50%, about 60% or about 70%, and ranges thereof, such as from about 30% to about 60%, from about 45% to about 65%, from 32% to 53%, or from 40% to 50%. Blend time is suitably, 5 min, 10 min, 15 min, 20 min, 30 min, 40 min, 50 min, 60 min, or more. Rotation rate is suitably, for instance, 2 rpm, 3 rpm, 4 rpm, 5 rpm, 6 rpm, 7 rpm, 8 rpm, 9 rpm or 10 rpm.

Excipient, lubricant and disintegrants are typically delumped by screening prior to blending. Screening methods are known to those skilled in the art.

In an example of one particular pre-blend aspect of the disclosure, excipient (e.g., MCC) and disintegrant (e.g., croscarmellose sodium) are delumped by screening and are combined with the active drug (e.g., divarasib adipate) in a blender, and the blender contents are blended for a blend time (e.g., 30 minutes) at a fixed rotation rate (e.g., 6 rpm). Lubricant (e.g., magnesium stearate) is delumped by screening and is added to a blender containing admixed excipient, disintegrant and active drug (e.g. divarasib adipate). The blender contents are blended for a blend time (e.g., 8 minutes) at a fixed rotation rate (e.g., 6 rpm) to form the pre-blend.

In another example of one pre-blend aspect of the disclosure, the active drug (e.g., divarasib adipate) is blended with a delumped disintegrant (e.g., croscarmellose sodium) for a blend time (e.g., 5 minutes at 6 rpm). Delumped excipient (e.g., MCC) is added to the blender and the contents are blended for a blend time (e.g., minutes at 6 rpm). Delumped lubricant (e.g., magnesium stearate) is added to the blender and the contents are blended for a blend time (e.g., 2 minutes).

Granulation and Sizing. Granulation and sizing may be achieved using any suitable means known to those skilled in the art. In some particular aspects of the disclosure, granulation and sizing comprises dry granulation, milling and screening (sieving). In some other aspects of the disclosure, dry granulation is roller compaction.

Granulation and sizing improves flow and compression characteristics of the admixture of active drug and excipients. Roller compaction is a process wherein pre-blend powder particles are made to adhere together resulting in larger, granular multi-particle entities. Roller compaction generally comprises three unit operations including a feeding system, a compaction unit and a milling/sieving unit. In the compaction unit, the pre-blend is compacted between counter-rotating rolls by application of a roller compaction force (expressed in kN/cm) to form a formed mass of compacted material, such as a ribbon or a sheet. The distance between the rolls is defined as the gap width. The formed ribbon of compacted material is processed in a size reduction unit by milling to form granules that are screened to produce a plurality of granules having a desired particle size distribution.

Roller compaction and milling equipment is available commercially from a number of manufacturers including Gerteis, Fitzpatrick®, Freund-Vector. Such equipment generally provides for control of roller compaction force, gap width, roller speed and feed rate. The roller surfaces may be smooth, knurled, or one roller surface may be smooth and the other roller surface may be knurled. In any of the various aspects, the pre-blend is charged to a roller compactor feed hopper. Roller compaction is performed at a specified force and gap size, and the process is preferably run under gap control. The formed ribbons are milled through a screen to produce granules. In some aspects of the disclosure, the screen is integral to the mill.

Without being bound to any particular theory, it is believed that the properties of the ribbon and granules formed therefrom are affected by the combination of roller compaction and milling variables including roller compaction force, gap width, material mass throughput, the screen size, and the uniformity and composition of the pre-blend. It is further believed that the properties of the formed ribbon (as influenced by gap size, roller compaction force, etc.) affects tablet appearance due, among other effects, to punch filming/sticking. It is further believed that the roller compaction variables may affect granule particle size distribution, granule density (thus, compressibility), and granule flow. Roller compaction gap size is believed to have an impact on cohesiveness of the final blend particles, with smaller gap size leading to granules with a greater tendency to stick. It is further believed that a smaller gap size may result in filming on the punches during tablet compression and the production of tablets with appearance defects. Roller compaction force is believed to influence the densification of the pre-blend during granulation, to affect granule properties, and to affect in vitro dissolution of the resulting tablets. Increasing roller compaction force may produce a final blend with better flowability, leading to reduced variability in main compression force and tablet weight, and increased control of uniformity of dosage units. Milling screen size impacts granule particle size distribution which may impact flowability and may impact the uniformity of the dosage units. For instance, poor flow could impact tableting die filling with concomitant tablet weight variation. It is believed that tablet disintegration is not generally affected by granule particle size in tablet embodiments wherein disintegrant is present both intra and extragranularly.

The combination of roller compaction force and gap size is believed to influence ribbon/granule density, especially as the gap is reduced, and to affect in vitro dissolution of the resulting tables. Gap size, along with roller compaction force, has also been observed to affect the sticking tendency of the final blend during tablet compression and the production of tablets with appearance defects.

In any of the various aspects of the disclosure, the gap size (gap width) is about 1 mm, about 2 mm, about 3 mm, about 4 mm, about 5 mm, or about 6 mm and ranges thereof, such as from about 1 mm to about 6 mm, about 2 mm to about 5 mm, from about 2 mm to about 4 mm, from about 3 mm to about 5 mm or from about 4 mm to about 5 mm. Based on experimental evidence to date, such gap sizes are generally sufficient to reduce sticking. The roller compaction force is about 1 kN/cm, about 2 kN/cm, about 3 kN/cm, about 4 kN/cm, about 5 kN/cm, about 6 kN/cm, about 7 kN/cm or about 8 kN/cm, and ranges thereof, such as from about 1 kN/cm to about 8 kN/cm, from about 2 kN/cm to about 5 kN/cm or from about 2 kN/cm to about 4 kN/cm.

In any of the various aspects of the disclosure, the milling screen size is 0.5 mm, 0.75 mm, 0.8 mm, 1.0 mm, 1.25 mm, 1.5 mm, 1.75 mm, 2.0 mm, 2.25 mm or 2.5 mm, and ranges thereof, such as from about 0.5 mm to about 2.5 mm, from about 0.5 mm to about 2.0 mm, from about 0.5 mm to about 1.5 mm, from about 0.5 mm to about 1.25 mm, from about 0.75 mm to about 2.5 mm, from about 0.75 mm to about 2.0 mm, from about 0.75 mm to about 1.5 mm, from about 0.75 mm to about 1.25 mm. In some particular aspects of the disclosure, a 1.0 mm milling screen is used.

Ribbon at-gap density (defined as ribbon throughput per operational time divided by the calculated volume per time) is the result of the combination of roller compaction parameters and is believed to be correlated with granule properties and with processability during tablet compression. Ribbon at-gap density is suitably about 0.85 g/mL, about 0.9 g/mL, about 0.95 g/mL, about 1.0 g/mL, about 1.05 g/mL, about 1.1 g/mL about 1.15 g/mL, about 1.2 g/mL, about 1.25 g/mL or about 1.3 g/mL and ranges thereof, such as from about 0.85 g/mL to about 1.3 g/mL, from about 0.9 g/mL to about 1.25 g/mL, from about 0.95 g/mL to about 1.2 g/mL. In some aspects of the disclosure low ribbon at-gap density of from about 0.85 g/mL to about 95 g/mL or from about 0.9 g/mL to about 0.95 g/mL is selected and controlled for. In some other aspects of the disclosure center point ribbon at-gap density of from about 0.95 g/mL to from about 1.1 g/mL, from about 0.95 g/mL to about 105 g/mL, from about 1 g/mL to about 1.10 g/mL or from about 1 g/mL to about 1.05 g/mL is selected and controlled for. In yet other aspects of the disclosure high ribbon at-gap density of from about 1.1 g/mL to about 1.3 g/mL, from about 1.1 g/mL to about 1.25 g/mL, from about 1.1 g/mL to about 1.2 g/mL, from about 1.1 g/mL to about 1.15 g/mL, from about 1.15 g/mL to about 1.3 g/mL, from about 1.15 g/mL to about 1.25 g/mL, or from about 1.15 g/mL to about 1.2 g/mL is selected and controlled for.

Final Blending. In the final blending step, granules formed by roller compaction and milling are charged to a blender and any extragranular portion of the disintegrant (e.g., croscarmellose sodium), excipient (e.g., MCC), and the lubricant (e.g., magnesium stearate) is added to the blender to form an admixture. The final blending step provide for an essentially homogeneous distribution of any external disintegrant, excipient, and lubricant and provides for acceptable processability during tablet compression. Suitable blenders and related process variables are described above.

In some aspects, the disintegrant and/or excipient are delumped prior to addition to the blender and the disintegrant and/or excipient are blended with the granules under a first set of blending conditions (e.g., for 2 minutes at 29 rpm). In a second final blending step, the lubricant is delumped and added to the blender and blended under a second set of blending conditions (e.g., for about 6 minutes at 29 rpm).

In any of the various aspects of the disclosure, the granule blend bulk density is about 0.4 g/mL, about 0.45 g/mL, about 0.5 g/mL, about 0.55 g/mL, about 0.6 g/mL, about 0.65 g/mL, about 0.7 g/mL or about 0.75 g/mL, and ranges thereof, such as from about 0.4 g/mL to about 0.75 g/mL, from about 0.45 g/mL to about 0.7 g/mL, or from 0.51 g/mL to 0.63 g/mL. The final blend is preferably easy or free flowing and has a flow function coefficient of at least 4.

Tableting. In the tableting step, a tableting die mold is filled with final blend material and the mixture is compressed to form a tablet core that is ejected. Suitable tablet presses are known in the art and are available commercially from, for instance, Korsch AG, Riva-Piccola, Fette, Bosch Packaging Technology, GEA and Natoli Engineering Company. Generally, each tablet is made by pressing the granules inside a die, made up of hardened steel. The die is a disc shape with a hole cut through its center. The powder is compressed in the center of the die by two hardened steel punches that fit into the top and bottom of the die thereby forming the tablet. Tablet compression may be done in two stages with the first, pre-compression, stage involving tamping down the powder and compacting the blend slightly prior to application of the main compression force for tablet formation. The tablet is ejected from the die after compression.

Main compression force affects tablet characteristics such as hardness and appearance. Main compression force further has an impact on sticking of the final blend to tablet tooling during compression, with increased force leading to reduced sticking and, hence, fewer tablets with appearance defects. Further, the compressibility of the final blend can impact the quality (such as the presence or lack of defects) of the resultant tablet core. Compression processing parameters, such as compression force and run time, can also have an impact. Final blend material attributes and compression process parameters may also have an impact on tablet weight variability and content. Further, variations in input final blend material attributes and compression processing parameters may have an impact on tablet weight variability, which is directly related to uniformity of dosage units. Moreover, quality attributes of the tablet core, such as tablet disintegration, hardness, friability, and porosity, are related to dissolution and impacted by compression processing parameters.

In some aspects of the disclosure, the compression force (tableting compaction force) is about 4 kN, about 5 kN, about 6 kN, about 7 kN, about 8 kN, about 9 kN, about 10 kN, about 11 kN, about 12 kN, about 13 kN, about 14 kN, about 15 kN, about 16 kN, about 17 kN, about 18 kN, about 19 kN or about 20 kN, and ranges thereof, such as from about 4 kN to about 20 kN, from about 14 kN to about 19 kN, from about 14 kN to about 18 kN, or from about 8 kN to about 13 kN. In some aspects of the disclosure, tablets comprising about 100 mg of the active drug may be formed at a compression force of from about 6 kN to about 9 kN. In other aspects of the disclosure, tablets comprising about 400 mg of the active drug may be formed at a compression force of from about 9 kN to about 12 kN.

Film Coating. The tablet cores are film-coated to ensure that tablets are essentially tasteless and odorless, and are easy to swallow. Film coating also prevents dust formation during packaging and ensures robustness during transportation. Film coating may suitably be done by methods known in the art such as by pan coating. Suitable coating equipment includes, without limitation, an O'Hara Labcoat™ system.

In some aspects of the disclosure, tablet cores are charged to a coating pan and warmed to a target temperature. The coating suspension is prepared to a target solids content. Once the tablets are within the target temperature range, drum rotation and spraying are runs at target rates designed to achieve predetermined weight gain of about 3 wt. %, about 4 wt. % or about 5 wt. %. Outlet air temperature is maintained in a range to ensure that the target product temperature is obtained throughout coating. Once spraying is complete, the coated tablets are dried and cooled down before discharging the film-coated tablets. A solid content of a coating suspension is suitably about 12 wt. %, about 13 wt. %, about 14 wt. %, about 15 wt. %, about 16 wt. %, about 17 wt. %, about 18 wt. %, about 19 wt. % or about 20 wt. %, and ranges thereof, such as from about 12 wt. % to about 20 wt. %, or from about 14 wt. % to about 20 wt. %. The coating spray rate per kg of tablet cores is suitably about 0.8, about 1, about 1.5, about 1.9 about 2, about 2.1, about 2.2, about 2.3, about 2.4 or about 2.5, and ranges thereof, such as from about 0.8 to about 2.5, or from about 1 to about 2.1. The coating temperature is suitably about 35° C., about 39° C., about 40° C., about 45° C., about 47° C., about 48° C., about 49° C., about 50° C. or about 55° C., and ranges thereof, such as from about 35° C. to about 50° C., or from about 39° C. to about 47° C. The pan rotational speed is suitably about 2 rpm, about 4 rpm, about 5 rpm, about 8 rpm, about 10 rpm, about 12 rpm, about 15 rpm or about 20 rpm, and ranges thereof, such as from about 2 to about 20 rpm, from about 4 to about 15 rpm, or from about 8 to about 12 rpm. The inlet air volume varies with the batch size and is suitably about 100 m³/h, about 300 m³/h, about 450 m³/h, about 600 m³/h, about 750 m³/h, about 1000 m³/h, about 1250 m³/h, or about 1500 m³/h, and ranges thereof, such as from about 100 m³/h to about 1500 m³/h, from about 300 to about 1500 m³/h, from about 450 to about 1200 m³/h, or from about 1000 to about 1250 m³/h.

Tablet Cores and Coated Tablets. In some aspects of the present disclosure the tablet core comprises the components and concentration ranges in wt. %, based on the core weight, as indicated in Table 1.

TABLE 1

| Component | 1^{st} Range | 2^{nd} Range | 3^{rd} Range |
|---|---|---|---|
| Active Drug | 1%-44% | 20%-44% | 30%-40% |
| (freebase equiv) | | | |
| Excipient | 15%-50% | 20%-45% | 30%-40% |
| Disintegrant | 2%-13% | 4%-11% | 6%-9% |
| Lubricant | 1%-3% | 1.5%-2.5% | 2%-2.5% |

In some aspects of the present disclosure, the tablet core comprises the components and concentration ranges in wt. %, based on the core weight, as indicated in Table 2.

TABLE 2

| Component | 1^{st} Range | 2^{nd} Range | 3^{rd} Range |
|---|---|---|---|
| divarasib adipate | 1%-44% | 20%-44% | 30%-40% |
| (freebase equiv) | | | |
| MCC | 15%-50% | 20%-45% | 30%-40% |
| NaCMC | 2%-13% | 4%-11% | 6%-9% |
| MgSt | 1%-3% | 1.5%-2.5% | 2%-2.5% |

MCC = microcrystalline cellulose;
NaCMC = croscarmellose sodium;
MgSt = magnesium stearate

In some aspects of the present disclosure, the tablet core comprises the components and concentration ranges in wt. %, based on the core weight, as indicated in Table 3.

TABLE 3

| | | | |
|---|---|---|---|
| Component | 1^{st} Range | 2^{nd} Range | 3^{rd} Range |

Intragranular

| | | | |
|---|---|---|---|
| Active Drug | 1%-44% | 20%-44% | 30%-40% |
| (freebase equiv) | | | |
| Excipient | 10%-30% | 17%-23% | 19%-21% |
| Disintegrant | 1%-5% | 2%-4% | 2.75%-3.25% |
| Lubricant | 0.1%-1.5% | 0.2%-1.25% | 0.5%-1.25% |

Extragranular

| | | | |
|---|---|---|---|
| Excipient | 5%-20% | 11%-19% | 14%-16% |
| Disintegrant | 1%-8% | 3%-6% | 4%-5% |
| Lubricant | 0.5%-2.5% | 0.75%-2% | 1.25%-1.5% |

In some aspects of the present disclosure, the tablet core comprises the components and concentration ranges in wt. %, based on the core weight, as indicated in Table 4.

TABLE 4

| | | | |
|---|---|---|---|
| Component | 1^{st} Range | 2^{nd} Range | 3^{rd} Range |

Intragranular

| | | | |
|---|---|---|---|
| divarasib adipate | 1%-44% | 20%-44% | 30%-40% |
| (freebase equiv) | | | |
| MCC | 10%-30% | 17%-23% | 19%-21% |
| NaCMC | 1%-5% | 2%-4% | 2.75%-3.25% |
| MgSt | 0.1%-1.5% | 0.2%-1.25% | 0.5%-1.25% |

Extragranular

| | | | |
|---|---|---|---|
| MCC | 5%-20% | 11%-19% | 14%-16% |
| NaCMC | 1%-8% | 3%-6% | 4%-5% |
| MgSt | 0.5%-2.5% | 0.75%-2% | 1.25%-1.5% |

MCC = microcrystalline cellulose;
NaCMC = croscarmellose sodium;
MgSt = magnesium stearate

In some aspects of the present disclosure, the tablet core comprises the components and concentration ranges in wt. %, based on the core weight, as indicated in Table 5.

TABLE 5

| | |
|---|---|
| Component | Amount (wt %) |

Intragranular

| | |
|---|---|
| divarasib adipate | 53.91%¹ |
| MCC | 20.84% |
| NaCMC | 3.0% |
| MgSt | 1.0% |

Extragranular

| | |
|---|---|
| MCC | 15.0% |
| NaCMC | 5.0% |
| MgSt | 1.25% |

¹corresponds to 40% freebase equivalence.
MCC = microcrystalline cellulose;
NaCMC = croscarmellose sodium;
MgSt = magnesium stearate

In some aspects of the present disclosure, the tablet cores are coated, and comprise the components and concentrations in wt. %, based on the core weight, as indicated in Table 6. The components and concentrations in wt % of an exemplary film coating composition is indicated in Table 7. Other standard film coatings may also be used, including but not limited to Opadry® products.

TABLE 6

| | |
|---|---|
| Component | Amount (wt %) |

Intragranular

| | |
|---|---|
| divarasib adipate | 53.91%¹ |
| MCC | 20.84% |
| NaCMC | 3.0% |
| MgSt | 1.0% |

Extragranular

| | |
|---|---|
| MCC | 15.0% |
| NaCMC | 5.0% |
| MgSt | 1.25% |
| Subtotal | 100% |
| Film Coating | 3.0% |
| Total | 103.0% |

MCC = microcrystalline cellulose;
NaCMC = croscarmellose sodium;
MgSt = magnesium stearate

TABLE 7

| Component | Amount (wt %) |
|---|---|
| Polyvinyl alcohol | 40% |
| Titanium dioxide | 25% |
| Macrogol/PEG 3350 | 20.2% |
| Talc | 14.8% |

Tablets and tablet cores of the present disclosure may have a tensile strength (TS) of from about 2.0 MPa to about 3.0 MPa, including about 2.0 MPa, about 2.5 MPa, and about 3.0 MPa. Tablets and tablet cores of the present disclosure may have a solid fraction (SF) of from about 0.80 to about 0.90.

In one embodiment, a tablet and tablet core is as set forth in one of the formulations provided in Table 8, 9, 10, 12, 13, 14, 15, or 16. In one such embodiment, a tablet and/or tablet core is as set forth in Table 8 herein. In another such embodiment, a tablet and/or tablet core is as set forth in Table 9 herein. In another such embodiment, a tablet and/or tablet core is as set forth in Table 10 herein. In another such embodiment, a tablet and/or tablet core is as set forth in Table 12 herein. In another such embodiment, a tablet and/or tablet core is as set forth in Table 13 herein. In another such embodiment, a tablet and/or tablet core is as set forth in Table 14 herein. In another such embodiment, a tablet and/or tablet core is as set forth in Table 15 herein. In another such embodiment, a tablet and/or tablet core is as set forth in Table 16 herein.

### EMBODIMENTS

Provided below are some exemplary embodiments of the invention.

Embodiment 1. A pharmaceutical dosage unit tablet core, the tablet core comprising:
(a) divarasib:
(b) a disintegrant at a content based on the pharmaceutical dosage unit tablet core weight of from about 2 wt % to about 13 wt %; and
(c) an excipient at a content based on the pharmaceutical dosage unit tablet core weight of from about 15 wt % to about 50 wt %.

Embodiment 2. The pharmaceutical dosage unit tablet core of embodiment 1, wherein the divarasib freebase content is up to about 44 wt %, based on the pharmaceutical dosage unit tablet core weight.

Embodiment 3. The pharmaceutical dosage unit tablet core of embodiment 1, wherein divarasib is divarasib adipate.

Embodiment 4. The pharmaceutical dosage unit tablet core of embodiment 3, wherein the divarasib adipate content is up to about 60 wt %, based on the pharmaceutical dosage unit tablet core weight.

Embodiment 5. The pharmaceutical dosage unit tablet core of any one of embodiments 1-4, wherein the disintegrant is selected from the group consisting of croscarmellose sodium (CCS), sodium starch glycolate (SSG), crospovidone (crosPVP), and combinations thereof.

Embodiment 6. The pharmaceutical dosage unit tablet core of embodiment 5, wherein the disintegrant is CCS.

Embodiment 7. The pharmaceutical dosage unit tablet core of any one of embodiments 1-6, wherein the excipient is selected from the group consisting of a starch, microcrystalline cellulose (MCC), and combinations thereof.

Embodiment 8. The pharmaceutical dosage unit tablet core of embodiment 7, wherein the excipient is MCC.

Embodiment 9. The pharmaceutical dosage unit tablet core of embodiment 8, wherein the MCC has an average particle size of about 20 uM to about 180 uM.

Embodiment 10. The pharmaceutical dosage unit tablet core of any one of embodiments 1-9, further comprising a lubricant at a content based on the pharmaceutical dosage unit tablet core weight of from about 1 wt % to about 3 wt %.

Embodiment 11. The pharmaceutical dosage unit tablet core of embodiment 10, wherein the lubricant is magnesium stearate.

Embodiment 12. The pharmaceutical dosage unit tablet core of any one of embodiments 1-11, wherein the pharmaceutical dosage unit tablet core has a tensile strength of from about 2.0 Mpa to about 3.0 Mpa.

Embodiment 13. The pharmaceutical dosage unit tablet core of any one of embodiments 1-12, wherein the pharmaceutical dosage unit tablet core has a solid fraction of from about 0.80 to about 0.90.

Embodiment 14. The pharmaceutical dosage unit tablet core of any one of embodiments 1-13, further comprising a film-coating on the surface of the tablet core.

Embodiment 15. The pharmaceutical dosage unit tablet core of any one of embodiments 1-14, wherein the pharmaceutical dosage unit tablet core has a disintegration time of about 15 minutes or less.

Embodiment 16. The pharmaceutical dosage unit tablet core of any one of embodiments 1-15, wherein the pharmaceutical dosage unit tablet core has a disintegration time of about 10 minutes or less.

Embodiment 17. The pharmaceutical dosage unit tablet core of any one of embodiments 1-16, wherein the pharmaceutical dosage unit tablet core has a disintegration time of about 5 minutes or less.

Embodiment 18. A process for preparing a pharmaceutical dosage unit tablet core, the process comprising:
(a) blending divarasib, an intragranular excipient, and an intragranular disintegrant to form a pre-blend;
(b) compacting the pre-blend by application of a compaction force;
(c) milling and screening the compacted pre-blend to form granules;
(d) blending the granules with an extragranular excipient and an extragranular disintegrant to form a granule blend; and
(e) tableting the granule blend by application of a tablet compaction force to form the pharmaceutical dosage unit tablet core.

Embodiment 19. The process of embodiment 18, wherein the intragranular excipient is selected from the group consisting of a starch, microcrystalline cellulose (MCC), and combinations thereof.

Embodiment 20. The process of embodiment 19, wherein the intragranular excipient is MCC.

Embodiment 21. The process of any one of embodiments 18-20, wherein the intragranular excipient content is from about 10 wt % to about 30 wt %, based on pharmaceutical dosage unit tablet core weight.

Embodiment 22. The process of embodiment 21, wherein the intragranular excipient content is from about 15 wt % to about 25 wt %, based on based on pharmaceutical dosage unit tablet core weight.

Embodiment 23. The process of embodiment 22, wherein the intragranular excipient content is from about 20 wt % to about 21 wt %, based on pharmaceutical dosage unit tablet core weight.

Embodiment 24. The process of any one of embodiments 18-23, wherein the intragranular disintegrant is selected from the group consisting of croscarmellose sodium (CCS), sodium starch glycolate (SSG), crospovidone (crosPVP), and combinations thereof.

Embodiment 25. The process of embodiment 24, wherein the intragranular disintegrant is CCS.

Embodiment 26. The process of any one of embodiments 18-25, wherein the intragranular disintegrant content is from about 1 wt % to about 5 wt %, based on pharmaceutical dosage unit tablet core weight.

Embodiment 27. The process of embodiment 26, wherein the intragranular disintegrant content is from about 2 wt % to about 4 wt %, based on pharmaceutical dosage unit tablet core weight.

Embodiment 28. The process of embodiment 27, wherein the intragranular disintegrant content is about 3 wt %, based on pharmaceutical dosage unit tablet core weight.

Embodiment 29. The process of any of embodiments 18-28, wherein the extragranular excipient is selected from the group consisting of a starch, microcrystalline cellulose (MCC), and combinations thereof.

Embodiment 30. The process of embodiment 29, wherein the extragranular excipient is MCC.

Embodiment 31. The process of any one of embodiments 18-30, wherein the extragranular excipient content is from about 5 wt % to about 20 wt %, based on pharmaceutical dosage unit tablet core weight.

Embodiment 32. The process of embodiment 31, wherein the extragranular excipient content is from about 10 wt % to about 20 wt %, based on based on pharmaceutical dosage unit tablet core weight.

Embodiment 33. The process of embodiment 32, wherein the extragranular excipient content is from about 15 wt % to about 20 wt %, based on pharmaceutical dosage unit tablet core weight.

Embodiment 34. The process of embodiment 33, wherein the extragranular excipient content is about 15 wt %, based on pharmaceutical dosage unit tablet core weight.

Embodiment 35. The process of any one of embodiments 18-34, wherein the extragranular disintegrant is selected from the group consisting of croscarmellose sodium (CCS), sodium starch glycolate (SSG), crospovidone (crosPVP), and combinations thereof.

Embodiment 36. The process of embodiment 35, wherein the extragranular disintegrant is CCS.

Embodiment 37. The process of any one of embodiments 18-36, wherein the extragranular disintegrant content is from about 1 wt % to about 8 wt %, based on pharmaceutical dosage unit tablet core weight.

Embodiment 38. The process of embodiment 37, wherein the extragranular disintegrant content is from about 4 wt % to about 5 wt %, based on pharmaceutical dosage unit tablet core weight.

Embodiment 39. The process of embodiment 38, wherein the extragranular disintegrant content is about 5 wt %, based on pharmaceutical dosage unit tablet core weight.

Embodiment 40. The process of any one of embodiments 18-39, further comprising, prior to compaction, blending the pre-blend with an intragranular lubricant.

Embodiment 41. The process of embodiment 40, wherein the intragranular lubricant is selected from the group consisting of magnesium stearate, sodium stearyl fumarate, stearic acid and combinations thereof.

Embodiment 42. The process of embodiment 41, wherein the intragranular lubricant is magnesium stearate.

Embodiment 43. The process of any one of embodiments 42-43, wherein the intragranular lubricant content is from about 1 wt % to about 3 wt %, based on the pharmaceutical dosage unit tablet core weight.

Embodiment 44. The process of embodiment 43, wherein the intragranular lubricant content is about 1 wt %, based on the pharmaceutical dosage unit tablet core weight.

Embodiment 45. The process of any one of embodiments 18-44, further comprising, prior to tableting, blending the granules, the extragranular excipient, and the extragranular disintegrant with an extragranular lubricant to form the granule blend.

Embodiment 46. The process of embodiment 45, wherein the extragranular lubricant is selected from the group consisting of magnesium stearate, sodium stearyl fumarate, stearic acid, and combinations thereof.

Embodiment 47. The process of embodiment 46, wherein the extragranular lubricant is magnesium stearate.

Embodiment 48. The process of any one of embodiments 45-47, wherein the extragranular lubricant content is from about 1 wt % to about 3 wt %, based on the pharmaceutical dosage unit tablet core weight.

Embodiment 49. The process of embodiment 48, wherein the extragranular lubricant content is from about 1 wt % to about 2 wt %, based on the pharmaceutical dosage unit tablet core weight.

Embodiment 50. The process of embodiment 49, wherein the extragranular lubricant content is about 1.25 wt %, based on the pharmaceutical dosage unit tablet core weight.

Embodiment 51. The process of any one of embodiments 18-50, wherein the divarasib freebase content is up to about 44 wt %, based on the pharmaceutical dosage unit tablet core weight.

Embodiment 52. The process of embodiment 51, wherein the divarasib freebase content is up to about 40 wt %, based on the pharmaceutical dosage unit tablet core weight.

Embodiment 53. The process of embodiment 52, wherein the divarasib freebase content is from about 1 wt % to about 44 wt %, based on the pharmaceutical dosage unit tablet core weight.

Embodiment 54. The process of embodiment 53, wherein the divarasib freebase content is from about 1 wt % to about 40 wt %, based on the pharmaceutical dosage unit tablet core weight.

Embodiment 55. The process of embodiment 54, wherein the divarasib freebase content is about 40 wt %, based on the pharmaceutical dosage unit tablet core weight.

Embodiment 56. The process of any one of embodiments 18-55, wherein divarasib is divarasib adipate.

Embodiment 57. The process of embodiment 56, wherein the divarasib adipate content is up to about 60 wt %, based on the pharmaceutical dosage unit tablet core weight.

Embodiment 58. The process of embodiment 56, wherein the divarasib adipate content is up to about 54 wt %, based on the pharmaceutical dosage unit tablet core weight.

Embodiment 59. The process of embodiment 56, wherein the divarasib adipate content is from about 1 wt % to about 60 wt %, based on the pharmaceutical dosage unit tablet core weight.

Embodiment 60. The process of embodiment 59, wherein the divarasib adipate content is from about 1 wt % to about 54 wt %, based on the pharmaceutical dosage unit tablet core weight.

Embodiment 61. The process of embodiment 59, wherein the divarasib adipate content is about 54 wt %, based on the pharmaceutical dosage unit tablet core weight.

Embodiment 62. The process of any one of embodiments 18-61, wherein the compaction force is from about 1 kN/cm to about 8 kN/cm.

Embodiment 63. The process of any one of embodiments 18-62, wherein the tableting compaction force is from about 4 kN to about 20 kN.

Embodiment 64. The process of any one of embodiments 18-63, wherein the pre-blend is compacted between at least two rotating rolls having a gap width of from about 1 mm to about 6 mm to form a ribbon, wherein the ribbon is milled and screened to form the granules.

Embodiment 65. The process of any one of embodiments 18-64, wherein the screen size is from about 0.5 mm to about 2.5 mm.

Embodiment 66. The process of any one of embodiments 18-65, further comprising applying a film coating on the surface of the pharmaceutical dosage unit tablet core.

Embodiment 67. The process of any one of embodiments 18-66, wherein the bulk density of the granule blend is from about 0.4 g/mL to about 0.75 g/mL.

Embodiment 68. The pharmaceutical dosage unit tablet core of any one of claims 1-17 or the process of any one of embodiments 18-67, wherein the pharmaceutical dosage unit tablet core comprises the formulation of Table 8.

Embodiment 69. The pharmaceutical dosage unit tablet core of any one of claims 1-17 or the process of any one of embodiments 18-67, wherein the pharmaceutical dosage unit tablet core comprises the formulation of Table 9.

Embodiment 70. The pharmaceutical dosage unit tablet core of any one of claims 1-17 or the process of any one of embodiments 18-67, wherein the pharmaceutical dosage unit tablet core comprises the formulation of Table 10.

Embodiment 71. The pharmaceutical dosage unit tablet core of any one of claims 1-17 or the process of any one of embodiments 18-67, wherein the pharmaceutical dosage unit tablet core comprises the formulation of Table 12.

Embodiment 72. The pharmaceutical dosage unit tablet core of any one of claims 1-17 or the process of any one of embodiments 18-67, wherein the pharmaceutical dosage unit tablet core comprises the formulation of Table 13.

Embodiment 73. The pharmaceutical dosage unit tablet core of any one of claims 1-17 or the process of any one of embodiments 18-67, wherein the pharmaceutical dosage unit tablet core comprises the formulation of Table 14.

Embodiment 74. The pharmaceutical dosage unit tablet core of any one of claims 1-17 or the process of any one of embodiments 18-67, wherein the pharmaceutical dosage unit tablet core comprises the formulation of Table 15.

Embodiment 75. The pharmaceutical dosage unit tablet core of any one of claims 1-17 or the process of any one of embodiments 18-67, wherein the pharmaceutical dosage unit tablet core comprises the formulation of Table 16.

Embodiment 76. The pharmaceutical dosage unit tablet core of any one of claims 1-17 or the process of any one of embodiments 18-67, wherein the pharmaceutical dosage unit tablet core comprises the formulation of Table 1.

Embodiment 77. The pharmaceutical dosage unit tablet core of any one of claims 1-17 or the process of any one of embodiments 18-67, wherein the pharmaceutical dosage unit tablet core comprises the formulation of Table 2.

Embodiment 78. The pharmaceutical dosage unit tablet core of any one of claims 1-17 or the process of any one of embodiments 18-67, wherein the pharmaceutical dosage unit tablet core comprises the formulation of Table 3.

Embodiment 79. The pharmaceutical dosage unit tablet core of any one of claims 1-17 or the process of any one of embodiments 18-67, wherein the pharmaceutical dosage unit tablet core comprises the formulation of Table 4.

Embodiment 80. The pharmaceutical dosage unit tablet core of any one of claims 1-17 or the process of any one of embodiments 18-67, wherein the pharmaceutical dosage unit tablet core comprises the formulation of Table 5.

Embodiment 81. The pharmaceutical dosage unit tablet core of any one of claims 1-17 or the process of any one of embodiments 18-67, wherein the pharmaceutical dosage unit tablet core comprises the formulation of Table 6.

The following examples are presented byway of illustration, not limitation.

### EXAMPLES

Various testing and analytical methods are described herein.

### High Shear Wet Granulation (HSWG)

Tablets were prepared using high shear wet granulation according to the following procedure:

All intragranular components were charged into a granulation bowl equipped with an impeller and a chopper. The powder blend was dry mixed, followed by water addition at a controlled rate using a peristaltic pump and spray nozzle. The resulting wet mass was passed through a #10 mesh screen (1.70 mm) and subject to fluid bed drying. The dried material was passed through #18 mesh screen (1.00 mm) or #35 mesh screen (0.50 mm) to form the “coarse” or “fine” granule, respectively.

### Dry Granulation (DG)

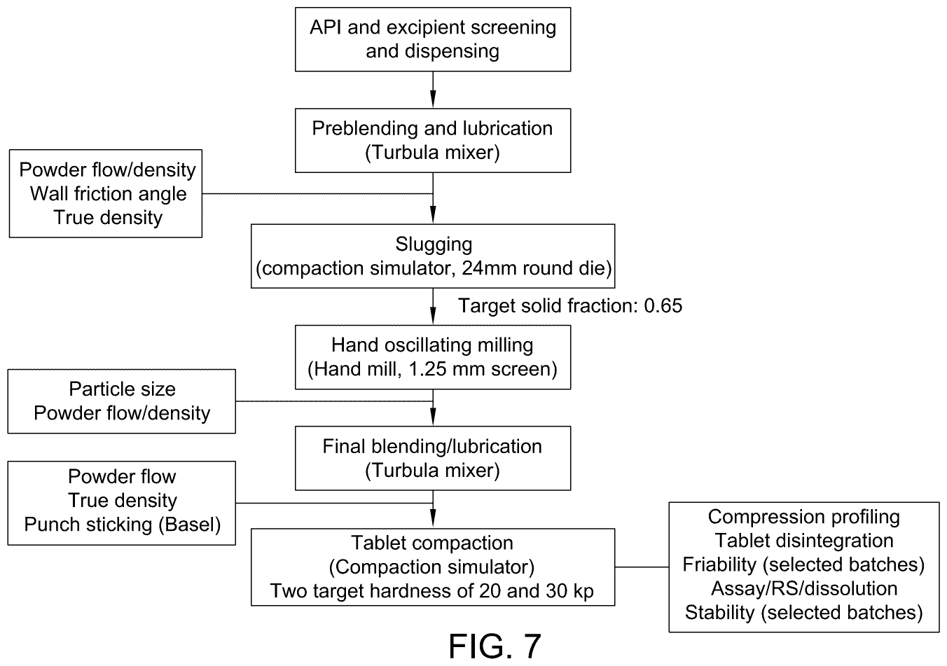

Tablets were prepared using dry granulation according to the following procedure. A flow chart illustrating the manufacturing process is set forth in FIG. 7.

API and excipient screening and dispensing: The active pharmaceutical ingredient (API) (i.e., divarasib adipate) and all excipients, with the exception of magnesium stearate, was screened through #18 mesh (screen size: 1 mm) sieve.

Magnesium stearate was screened through #30 mesh (screen size: 0.60 mm) sieve prior to use.

Powder blending (including preblending and final blending): All powder blending processes were conducted by mixing the powder in an appropriately sized bottle using a Turbula® mixer at 34 rpm.

Drygranulation (Slugging): The dry granulation of the intra-granular blend was conducted using a compaction simulator. Specifically, a 24-mm flat round punch-die set was employed for the dry granulation. The powder was consolidated by following a tailor-made punch movement profile simulating the powder densification within the nip region of an at-scale roller compactor. The target solid fraction of the resulting compact was achieved by adjusting the powder fill weight.

Milling: The milling of the compact resulting from the dry granulation was performed using a manual oscillating mill.

Tablet compression: The tablet compression was conducted using the compaction simulator. A punch-die set for the 19.0×9.50 mm, capsule-shape, plain concave tablet was chosen in the study. The target tablet weight for the Example 7 study is 1000 mg. The tablet compression followed the punch movement profile of a rotary press operated at the turret speed of 30 rpm.

### Film Coating

Unless otherwise indicated, film coated tablets are prepared using a pan coater equipped with an 8.5-in perforated pan. The coating suspension, with the solid fraction of 0.15, was spray onto the tablets at the rate of 3 g/min. The air volume and exhaust temperature were kept at 100 cfm and 42° C., respectively. Additional drying was implemented after the spraying was completed. The target coating weight gain was 3%

### Disintegration Time (DT)

Tablet disintegration time was determined using the procedure described in the general chapter “Disintegration” described in US Pharmacopeia (USP)<701>. Specifically, unless indicated otherwise, the following methodology was used in the examples described herein:

The tablet disintegration experiments were performed using a tablet disintegration tester consisting of a basket-rack assembly with 6 transparent tubes and a 1000-mL beaker. The bottom plates of the tubes are woven stainless steel wire cloth with the aperture of 2 mm. Tablets were placed in the tubes and subjected to raising and lowering movement in the immersion fluid (water) at 37° C. A tablet is considered fully disintegrated when no residual powder is left on the bottom plate.

### Dissolution

Unless otherwise indicated, the dissolution tests were performed in a USP II Apparatus with 900 mL of 50 mM citrate buffer (pH 3.0) at a paddle speed of 75 RPM at 37° C.

### Hardness

Tablet hardness testing is known in the art and is a measure of the breaking point and structural integrity of a tablet. In one compression test method, an aligned tablet is compressed in a testing apparatus with increasing force continually applied until the tablet fractures thereby indicating the hardness.

### Example 1

Example 1 evaluated the effect of different disintegrants on tablet disintegration time. The tablets were prepared using HSWG according to the methods described herein and the formulations set forth in Table 8 below.

TABLE 8

| | | |
|---|---|---|
| Material | Weight (%) | Quantity (g) |

Intragranular

| | | |
|---|---|---|
| divarasib adipate | 51.2² | 2.56 |
| MCC Avicel PH 101 | 38.3 | 1.92 |
| Binder¹ | 4.0 | 0.20 |
| Disintegrant | 3.0 | 0.15 |

Extragranular

| | | |
|---|---|---|
| Disintegrant | 3.0 | 0.15 |
| Mg Stearate | 0.5 | 0.03 |

¹Hydroxypropyl cellulose (Klucel ™ EXF)
²40% by free base (FB) equivalence, conversion factor of 1.28

The disintegrants tested were croscarmellose sodium (“NaCMC”, disintegrant 1), crospovidone (“crosPVP”, disintegrant 2), and sodium starch glycolate (“SSG”, disintegrant 3).

The tablets were compressed using an 8 mm plain standard concave tool using a compression force of 5.9 kN, and had a target weight of 200 mg. The disintegration time of the tablets was tested using the method described herein. The tablet properties and results are set forth in Table 9.

TABLE 9

| | Lot 1 (NaCMC) | | Lot 2 (crosPVP) | | Lot 3 (SSG) | |
|---|---|---|---|---|---|---|
| | Tablet | Tablet | Tablet | Tablet | Tablet | Tablet |
| | 1 | 2 | 1 | 2 | 1 | 2 |
| Weight | 206.6 | n/a | 201.4 | 201.9 | 209.3 | 211.0 |
| (mg) | | | | | | |
| Thickness | 4.43 | n/a | 4.42 | 4.46 | 4.36 | 4.39 |
| (mm) | | | | | | |
| Hardness | 8.9 | n/a | 10.7 | 10.1 | 15.0 | 15.0 |
| (kp) | | | | | | |
| Disintegration | 4.6 | 5.1 | 15-16 | 15-16 | 23-24 | 23-24 |
| time (min) | | | | | | |

All tablets eroded from the surface, and no obvious expansion of the tablets was observed. The results demonstrate that NaCMC was superior to SSG and the neutral disintegrant (crosPVP) at improving disintegration time. Water penetration into the tablet was determined to be the rate limiting factor for tablet disintegration.

### Example 2

Example 2 evaluated the effect of different extragranular (ExG) excipients on tablet disintegration time. The tablets were prepared using HSWG according to the methods described herein and the formulations set forth in Table 10 below. The formulation of a control tablet is set forth in Table 11.

TABLE 10

| | | |
|---|---|---|
| Material | Weight (%) | Quantity (g) |

Intragranular

| | | |
|---|---|---|
| divarasib adipate | 34.6%² | 1.92 |
| MCC Avicel PH 101 | 46.9% | 2.61 |
| Binder¹ | 0.9% | 0.05 |
| Disintegrant (NaCMC) | 3.6% | 0.20 |

Extragranular

| | | |
|---|---|---|
| Disintegrant (NaCMC) | 3.6% | 0.20 |
| Excipient | 10% | 0.56 |
| Mg Stearate | 0.4% | 0.02 |

¹Hydroxypropyl cellulose (Klucel ™ EXF)
²27% by free base (FB) equivalence, conversion factor of 1.28

TABLE 11
control

| | | |
|---|---|---|
| Material | Weight (%) | Quantity (g) |

Intragranular

| | | |
|---|---|---|
| divarasib adipate | 38.4%² | 1.92 |
| MCC Avicel PH 101 | 52.1% | 2.61 |
| Binder¹ | 1.0% | 0.05 |
| Disintegrant (NaCMC) | 4.0% | 0.20 |

Extragranular

| | | |
|---|---|---|
| Disintegrant (NaCMC) | 4.0% | 0.20 |
| Mg Stearate | 0.5% | 0.02 |

¹Hydroxypropyl cellulose (Klucel ™ EXF)
²27% by free base (FB) equivalence, conversion factor of 1.28

Tablets were compressed to a tensile strength of 2.5 MPa. The extragranular excipients were chosen to evaluate the ability of water-soluble excipients, less compressible excipients, and highly hydrophilic excipients to enhance the rate of water penetration into the tablet. The extragranular excipients tested were:

(1) MCC (Avicel PH 101)—percolating matrix of hydrophilic polymers to enhance water wicking; (2) Spray dried lactose—highly water-soluble excipient; (3) Mannitol—highly water-soluble excipient; and (4) Dicalcium phosphate—incompressible excipient.

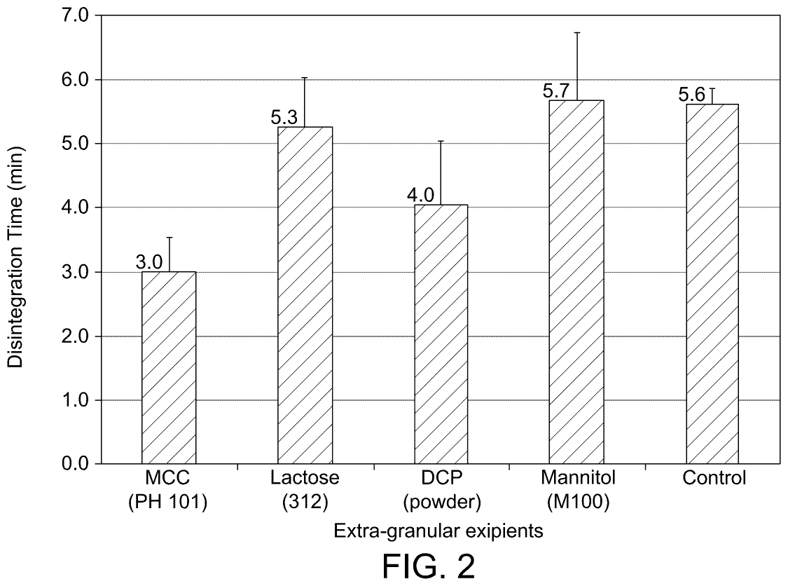

The results are shown in FIG. 2. As can be seen from these results, inclusion of a highly hydrophilic excipient such as MCC reduced disintegration time in comparison to other extragranular excipients.

### Example 3

Example 3 evaluated the effect of extra-granular microcrystalline cellulose (MCC) content on tablet disintegration time. The tablets were prepared using HSWG according to the methods described herein and the formulations set forth in Table 12 below.

TABLE 12

| | | | |
|---|---|---|---|
| Component | Tablet 1 | Tablet 2 | Tablet 3 |

Intragranular

| | | | |
|---|---|---|---|
| divarasib adipate (wt %) | 43.2%¹ | 43.2%¹ | 43.2%¹ |
| MCC Avicel PH 101 (wt %) | 36.3% | 31.3% | 26.3% |
| Hydroxypropyl cellulose | 2.0% | 2.0% | 2.0% |
| (Klucel ™ EXF) | | | |
| NaCMC (wt %) | 4.0% | 4.0% | 4.0% |

Extragranular

| | | | |
|---|---|---|---|
| MCC Avicel PH 101 (wt %) | 10.0% | 15.0% | 20.0% |
| NaCMC (wt %) | 4.0% | 4.0% | 4.0% |
| Mg Stearate (wt %) | 0.5% | 0.5% | 0.5% |

¹35% by free base (FB) equivalence, conversion factor of 1.235

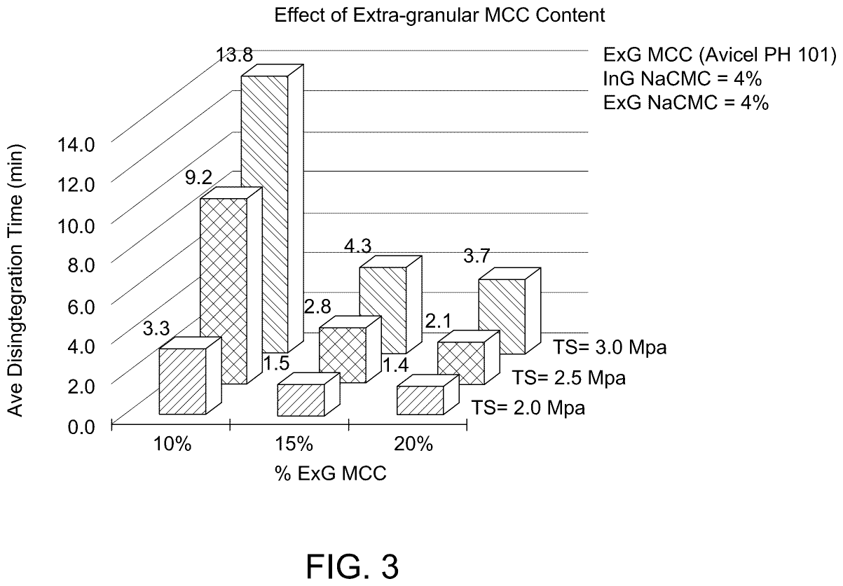

The tablets were compressed to a tensile strength of 2.0 MPa, 2.5 MPa, or 3.0 MPa. The average disintegration time (3 samples) was determined. The results are set forth in FIG. 3.

The tablets containing 15% extragranular MCC had a substantially faster disintegration time than tablets containing only 10% extragranular MCC. A slight improvement in disintegration time was observed when the extragranular MCC content was raised to 20%. These results suggest that inclusion of a sufficient amount of extragranular MCC is important for improving disintegration time.

### Example 4

Example 4 evaluated the effect of intragranular and extragranular croscarmellose sodium (NaCMC) content on tablet disintegration time. The tablets were prepared using HSWG according to the methods described herein and the formulations set forth in Table 13 below.

TABLE 13

| | | | | |
|---|---|---|---|---|
| Component | Tablet 2 | Tablet 4 | Tablet 5 | Tablet 6 |

Intragranular

| | | | | |
|---|---|---|---|---|
| divarasib adipate (wt %) | 43.2%¹ | 43.2%¹ | 43.2%¹ | 43.2%¹ |
| MCC Avicel PH 101 (wt %) | 31.3% | 31.3% | 33.3% | 33.3% |
| Hydroxypropyl cellulose | 2.0% | 2.0% | 2.0% | 2.0% |
| (Klucel ™ EXF) | | | | |
| NaCMC (wt %) | 4.0% | 4.0% | 2.0% | 2.0% |

Extragranular

| | | | | |
|---|---|---|---|---|
| MCC Avicel PH 101 (wt %) | 15.0% | 17.0% | 15.0% | 17.0% |
| NaCMC (wt %) | 4.0% | 2.0% | 4.0% | 2.0% |
| Mg Stearate (wt %) | 0.5% | 0.5% | 0.5% | 0.5% |

¹35% by free base (FB) equivalence, conversion factor of 1.235

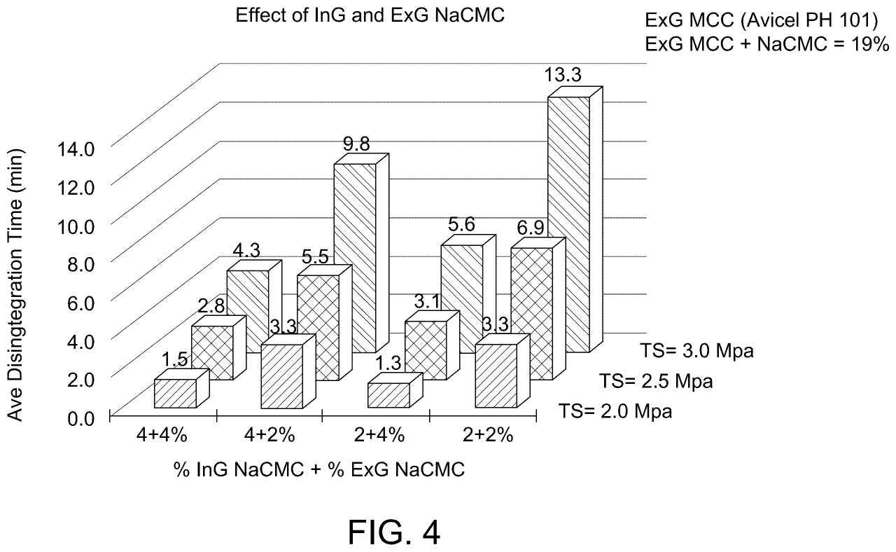

The tablets were compressed to a tensile strength of 2.0 MPa, 2.5 MPa, or 3.0 MPa. The average disintegration time was determined. The results are set forth in FIG. 4.

As can be seen from these results, tablets containing 4% extragranular NaCMC had a faster average disintegration time (3 samples) than tablets containing only 2% extragranular MCC.

### Example 5

Example 5 evaluated the effect of different grades of extracellular MCC on tablet disintegration time. The tablets were prepared using HSWG according to the methods described herein and the formulations set forth in Table 14 below.

TABLE 14

| | | |
|---|---|---|
| Tablet 1 | Tablet 7 | Tablet 8 |

Intragranular

| | | | |
|---|---|---|---|
| divarasib adipate (wt %) | 43.2%¹ | 43.2%¹ | 44.8%² |
| MCC Avicel PH 101 (wt %) | 36.3% | 36.3% | 34.7% |
| Hydroxypropyl cellulose | 2.0% | 2.0% | 2.0% |
| (Klucel ™ EXF) | | | |
| NaCMC (wt %) | 4.0% | 4.0% | 4.0% |

Extragranular

| | | | |
|---|---|---|---|
| MCC Avicel PH 101 (wt %) | 10.0% | 0 | 0 |
| MCC Avicel PH 200 (wt %) | 0 | 10.0% | 0 |
| MCC Avicel PH 105 (wt %) | 0 | 0 | 10.0% |
| NaCMC (wt %) | 4.0% | 4.0% | 4.0% |
| Mg Stearate (wt %) | 0.5% | 0.5% | 0.5% |

¹35% by free base (FB) equivalence, conversion factor of 1.235
²35% by free base (FB) equivalence, conversion factor of 1.28

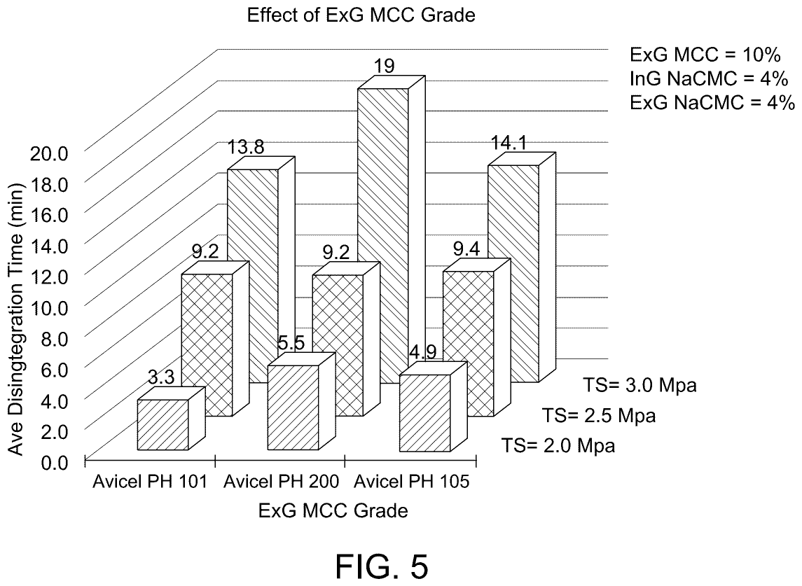

The tablets were compressed to a tensile strength of 2.0 MPa, 2.5 MPa, or 3.0 MPa. The average disintegration time (3 samples) was determined. The results are set forth in FIG. 5.

Tablets formulated with MCC Avicel PH 101 as the extragranular excipient had a faster average disintegration time than tablets prepared with MCC Avicel PH 200 or MCC Avicel PH 105.

### Example 6

Example 6 compared the disintegration times of tablets formulated by dry granulation (DG) to that of tablets formulated by HSWG. The tablets were prepared using HSWG or DG according to the methods described herein and the formulations set forth in Table 15 below.

TABLE 15

| | | | |
|---|---|---|---|
| Component | Tablet 2 | Tablet 9 | Tablet 10 |

Intragranular

| | | | |
|---|---|---|---|
| divarasib adipate (wt %) | 43.2%¹ | 43.2%¹ | 43.2%¹ |
| MCC Avicel PH 101 (wt %) | 31.3% | 0 | 0 |
| MCC Avicel PH 102 (wt %) | 0 | 32.8% | 32.8% |
| Hydroxypropyl cellulose | 2.0% | 0 | 0 |
| (Klucel ™ EXF) | | | |
| NaCMC (wt %) | 4.0% | 4.0% | 4.0% |
| Mg Stearate (wt %) | 0 | 0.5% | 0.5% |

Extragranular

| | | | |
|---|---|---|---|
| MCC Avicel PH 101 (wt %) | 15.0% | 15.0% | 15.0% |
| NaCMC (wt %) | 4.0% | 4.0% | 4.0% |
| Mg Stearate (wt %) | 0.5% | 0.5% | 0.5% |
| Slug solid fraction (SF) | n/a | 0.60 | 0.65 |

¹35% by free base (FB) equivalence, conversion factor of 1.235

Tablet 2 was prepared using HSWG and Tablets 10 and 11 were prepared using DG. The tablets had a tensile strength of 2.0 MPa, 2.5 MPa, or 3.0 MPa.

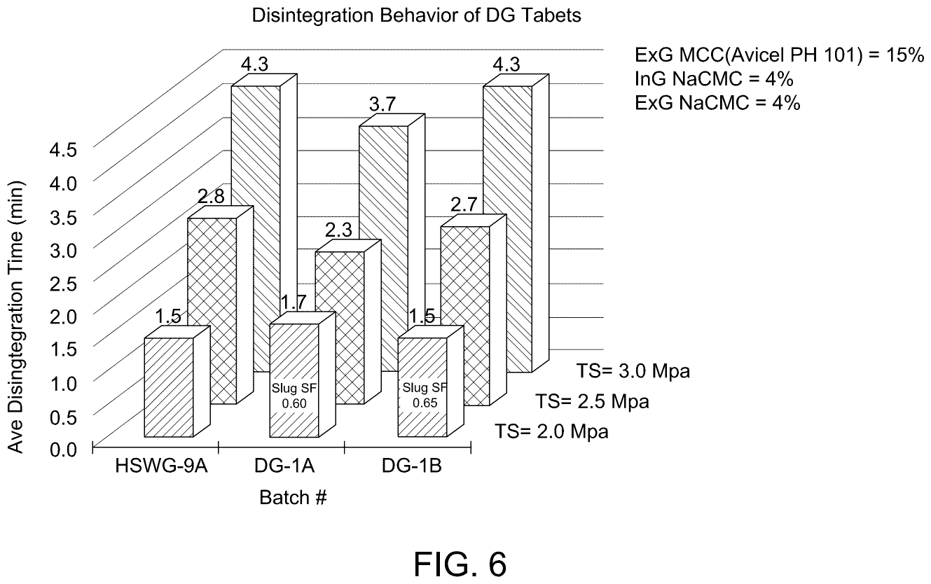

The average disintegration time (3 samples) was determined. The results are set forth in FIG. 6.

As can be seen from these results, tablets prepared using DG and tablets prepared using HSWG had similar disintegration behavior.

### Example 7

Example 7 evaluated the mechanical properties, disintegration times, and dissolution of various tablets formulated by dry granulation (DG). All formulations contain 15% MCC (Avicel PH 101) to aid tablet disintegration. The tablets were prepared using DG according to the methods described herein and the formulations set forth in Table 16 below.

TABLE 16

| | | | % InG | | | % ExG | | | Target |
|---|---|---|---|---|---|---|---|---|---|
| | Batch | % Drug | MCC | % InG | % InG | MCC | % ExG | % ExG | hardness |
| Run | # | (salt)** | (PH 102) | NaCMC | MgSt | (PH 101) | MgSt | NaCMC | tablet (kp) |
| 1 | DG-7* | 47.17% | 29.08% | 3.00% | 0.75% | 15.00% | 1.00% | 4.00% | 30 |
| 2 | | | | | | | | | 20 |
| 3 | DG-8 | 57.28% | 17.72% | 3.00% | 0.75% | 15.00% | 1.25% | 5.00% | 30 |
| 4 | | | | | | | | | 20 |
| 5 | DG-9 | 37.06% | 39.94% | 3.00% | 0.75% | 15.00% | 1.25% | 3.00% | 30 |
| 6 | | | | | | | | | 20 |
| 7 | DG-10 | 37.06% | 38.44% | 3.00% | 0.75% | 15.00% | 0.75% | 5.00% | 30 |
| 8 | | | | | | | | | 20 |
| 9 | DG-11 | 57.28% | 20.22% | 3.00% | 0.75% | 15.00% | 0.75% | 3.00% | 30 |
| 10 | | | | | | | | | 20 |
| 11 | DG-12 | 57.28% | 18.22% | 3.00% | 0.75% | 15.00% | 0.75% | 5.00% | 30 |
| 12 | | | | | | | | | 20 |
| 13 | DG-13 | 37.06% | 40.44% | 3.00% | 0.75% | 15.00% | 0.75% | 3.00% | 30 |
| 14 | | | | | | | | | 20 |
| 15 | DG-14 | 37.06% | 37.94% | 3.00% | 0.75% | 15.00% | 1.25% | 5.00% | 30 |
| 16 | | | | | | | | | 20 |
| 17 | DG-15 | 57.28% | 19.72% | 3.00% | 0.75% | 15.00% | 1.25% | 3.00% | 30 |
| 18 | | | | | | | | | 20 |
| 19 | DG-16 | 47.17% | 29.08% | 3.00% | 0.75% | 15.00% | 1.00% | 4.00% | 30 |
| 20 | | | | | | | | | 20 |

*The API content (by salt) was calculated based on the reported assay value (assay as-is % wt: 74.2%).

All batches contained 15% extra-granular (ExG) MOO (Avicel PH 101), 0.75% intra-granular (InG) Mg stearate, and 3.0% intra-granular croscarmellose sodium (NaCMO). Intra-granular MOO (Avicel PH 102) was also incorporated to compensate for the variation of other formulation components. All batches used API batch K2, the properties of which are shown below in Table 17.

TABLE 17
Batch K2 properties.

| | |
|---|---|
| Bulk density (g/mL) | 0.28 |
| Tapped density (g/mL) | 0.37 |
| PSD d10 (um) | 8.0 |
| PSD d50 (um) | 67 |
| PSD d90 (um) | 105 |

The manufacturing of the formulation batches was carried out in a material-sparing manner, by employing small-scale, emulative equipment. The batch sizes of the batches were 80-120 g. The manufacturing process for all tablet batches is shown in FIG. 7.

### Results and Discussion

Tablet Characterization: Tablet compression was carried out using a compaction simulator at a targeted tablet weight of 1000 mg as described herein. For each batch, a compression profiling was performed and the compression forces giving rise to tablets with hardnesses of 20 kp and 30 kp were identified. Following the compression profiling, additional tablets at the target hardness of 20 kp and 30 kp were produced for further analysis.

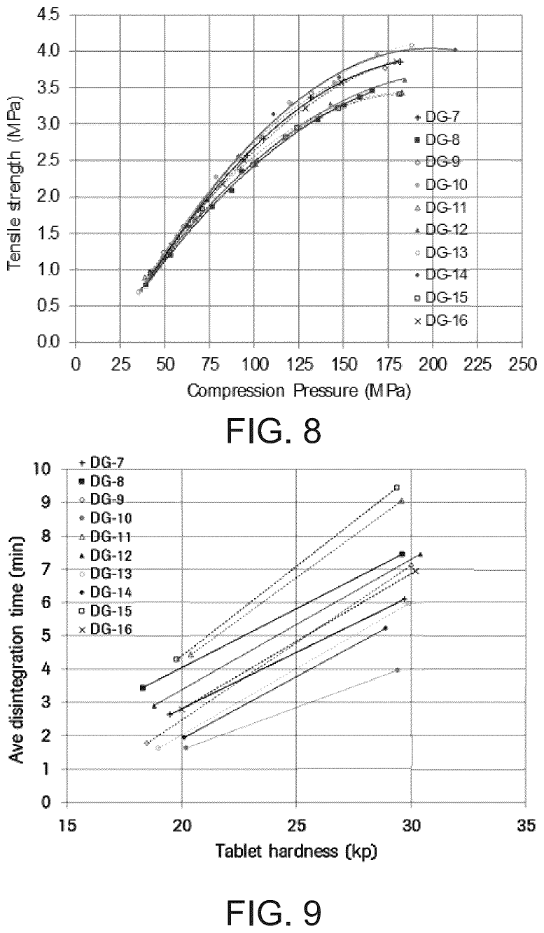

The tabletability profiles of all batches are shown in FIG. 8. The profiles indicate that divarasib formulations exhibit excellent tabletability in general. A tablet tensile strength of 2.0 MPa can be attained with a compression pressure of less than 100 MPa for all batches.

As shown in FIG. 8, slightly superior tabletability can be achieved for formulations containing lower drug content. No impact of the extra-granular magnesium stearate or NaCMC contents on tabletability was observed. Additional tablet characterization data for the batches, including the compression pressures leading to the tablet hardnesses of 20 and 30 kp, and tablet disintegration and dissolution data, is shown in Table 18.

TABLE 18

| | | Compression | | Disintegration | % dissolved | % dissolved |
|---|---|---|---|---|---|---|
| | Batch | pressure | Solid | time | 15 | 30 |
| Run | # | (MPa) | fraction | (min) | min | min |
| 1 | DG-7 | 60.2 | 0.781 | 2.8 | 98 | 104 |
| 2 | | 94.3 | 0.835 | 6.2 | 90 | 104 |
| 3 | DG-8 | 65.2 | 0.793 | 4.0 | 93 | 101 |
| 4 | | 105.5 | 0.858 | 7.6 | 75 | 99 |
| 5 | DG-9 | 60.2 | 0.782 | 2.5 | 95 | 98 |
| 6 | | 95.0 | 0.845 | 7.1 | 92 | 98 |
| 7 | DG-10 | 57.7 | 0.762 | 1.6 | 99 | 103 |
| 8 | | 86.3 | 0.821 | 4.1 | 97 | 105 |
| 9 | DG-11 | 61.4 | 0.793 | 4.2 | 88 | 99 |
| 10 | | 101.8 | 0.856 | 9.3 | 75 | 96 |
| 11 | DG-12 | 62.1 | 0.781 | 3.4 | 93 | 102 |
| 12 | | 103.6 | 0.844 | 7.3 | 81 | 97 |
| 13 | DG-13 | 58.3 | 0.760 | 2.0 | 104 | 106 |
| 14 | | 91.9 | 0.824 | 6.0 | 95 | 105 |
| 15 | DG-14 | 60.8 | 0.768 | 1.9 | 99 | 102 |
| 16 | | 90.6 | 0.832 | 5.6 | 93 | 101 |
| 17 | DG-15 | 62.1 | 0.788 | 4.4 | 94 | 101 |
| 18 | | 103.0 | 0.853 | 9.8 | 76 | 100 |
| 19 | DG-16 | 60.8 | 0.779 | 2.8 | 96 | 101 |
| 20 | | 97.4 | 0.840 | 6.9 | 88 | 100 |

The effect of drug, magnesium stearate, and NaCMC contents on the tablet disintegration time is clearly demonstrated in FIG. 9. The most pronounced deleterious effect arises from the high drug content, followed by low NaCMC content, and subsequently high Mg stearate content. The data indicates that the incorporation of high amount of extra-granular NaCMC (up to 5% in the study) can facilitate tablet disintegration, especially for tablets possessing high drug content and produced with a high compression pressure. Overall, including 15% extra-granular MOO and high extra-granular NaCMO content, can give rise to acceptable disintegration behavior of tablets.

The dissolution data of the tablets is presented in Table 18. For all tablets, the dissolution test was performed in USP apparatus 2, in 900 mL 50 mM citrate buffer (pH 3.0) and with the paddle speed of 75 RPM. Some effect of tablet hardness and drug content can be observed concerning the 15 min data point. Greater tablet hardness and high drug content resulted in slower dissolution, whereas the extra-granular magnesium stearate and NaCMC content showed no effect. However, at the 30 min data point, all batches exhibit >95% dissolution (Table 18). This data suggest that although changes of the formulation present in the study may have an impact on the dissolution at the early time point, the overall risk of dissolution retardation for the divarasib formulations is low, and an immediate release profile may be achieved.

This written description uses examples to disclose the invention, including the best mode, and also to enable any person skilled in the art to practice the invention, including making and using any devices or systems and performing any incorporated processes or methods. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal languages of the claims.

1. A pharmaceutical dosage unit tablet core, the tablet core comprising:
(a) divarasib:
(b) a disintegrant selected from the group consisting of croscarmellose sodium (CCS), sodium starch glycolate (SSG), crospovidone (crosPVP), and combinations thereof at a content based on the pharmaceutical dosage unit tablet core weight of from about 2 wt % to about 13 wt %; and
(c) an excipient selected from the group consisting of a starch, microcrystalline cellulose (MCC), and combinations thereof at a content based on the pharmaceutical dosage unit tablet core weight of from about 15 wt % to about 50 wt %.

2. The pharmaceutical dosage unit tablet core of claim 1, wherein the divarasib freebase content is up to about 44 wt %, based on the pharmaceutical dosage unit tablet core weight.

3. (canceled)

4. The pharmaceutical dosage unit tablet core of claim 1, wherein divarasib is divarasib adipate at a content of about 1 wt % to about 60 wt %, based on the pharmaceutical dosage unit tablet core weight.

5. (canceled)

6. The pharmaceutical dosage unit tablet core of claim 1, wherein the disintegrant is CCS.

7. (canceled)

8. The pharmaceutical dosage unit tablet core of claim 1, wherein the excipient is MCC.

9. (canceled)

10. The pharmaceutical dosage unit tablet core of claim 1, further comprising magnesium stearate at a content based on the pharmaceutical dosage unit tablet core weight of from about 1 wt % to about 3 wt %.

11. (canceled)

12. The pharmaceutical dosage unit tablet core of claim 1, wherein the pharmaceutical dosage unit tablet core has a tensile strength of from about 2.0 Mpa to about 3.0 Mpa and a solid fraction of from about 0.80 to about 0.90.

13.-14. (canceled)

15. The pharmaceutical dosage unit tablet core of claim 1, wherein the pharmaceutical dosage unit tablet core has a disintegration time of about 15 minutes or less.

16. The pharmaceutical dosage unit tablet core of claim 1, wherein the pharmaceutical dosage unit tablet core has a disintegration time of about 10 minutes or less.

17. The pharmaceutical dosage unit tablet core of claim 1, wherein the pharmaceutical dosage unit tablet core has a disintegration time of about 5 minutes or less.

18. A process for preparing a pharmaceutical dosage unit tablet core, the process comprising:
(a) blending divarasib, an intragranular excipient selected from the group consisting of a starch, microcrystalline cellulose (MCC), and combinations thereof and comprising about 10 wt % to about 30 wt %, based on pharmaceutical dosage unit tablet core weight, and an intragranular disintegrant selected from the group consisting of croscarmellose sodium (CCS), sodium starch glycolate (SSG), crospovidone (crosPVP), and combinations thereof and comprising about 1 wt % to about 5 wt %, based on pharmaceutical dosage unit tablet core weight to form a pre-blend;
(b) compacting the pre-blend by application of a compaction force;
(c) milling and screening the compacted pre-blend to form granules;
(d) blending the granules with an extragranular excipient selected from the group consisting of a starch, microcrystalline cellulose (MCC), and combinations thereof and comprising about 5 wt % to about 20 wt %, based on pharmaceutical dosage unit tablet core weight and an extragranular disintegrant selected from the group consisting of croscarmellose sodium (CCS), sodium starch glycolate (SSG), crospovidone (crosPVP), and combinations thereof comprising about 1 wt % to about 8 wt %, based on pharmaceutical dosage unit tablet core weight to form a granule blend; and
(e) tableting the granule blend by application of a tablet compaction force to form the pharmaceutical dosage unit tablet core.

19. (canceled)

20. The process of claim 18, wherein the intragranular excipient is MCC.

21. (canceled)

22. The process of claim 18, wherein the intragranular excipient content is from about 15 wt % to about 25 wt %, based on based on pharmaceutical dosage unit tablet core weight.

23.-24. (canceled)

25. The process of claim 18, wherein the intragranular disintegrant is CCS.

26. (canceled)

27. The process of claim 18, wherein the intragranular disintegrant content is from about 2 wt % to about 4 wt %, based on pharmaceutical dosage unit tablet core weight.

28.-29. (canceled)

30. The process of claim 18, wherein the extragranular excipient is MCC.

31.-33. (canceled)

34. The process of claim 18, wherein the extragranular excipient content is about 15 wt %, based on pharmaceutical dosage unit tablet core weight.

35. (canceled)

36. The process of claim 18, wherein the extragranular disintegrant is CCS.

37.-38. (canceled)

39. The process of claim 18, wherein the extragranular disintegrant content is about 5 wt %, based on pharmaceutical dosage unit tablet core weight.

40. The process of claim 18, further comprising, prior to compaction, blending the pre-blend with an intragranular lubricant selected from the group consisting of magnesium stearate, sodium stearyl fumarate, stearic acid and combinations thereof comprising about 1 wt % to about 3 wt %, based on the pharmaceutical dosage unit tablet core weight.

41. (canceled)

42. The process of claim 40, wherein the intragranular lubricant is magnesium stearate.

43.-44. (canceled)

45. The process of claim 18, further comprising, prior to tableting, blending the granules, the extragranular excipient, and the extragranular disintegrant with an extragranular lubricant to form the granule blend selected from the group consisting of magnesium stearate, sodium stearyl fumarate, stearic acid, and combinations thereof comprising about 1 wt % to about 3 wt %, based on the pharmaceutical dosage unit tablet core weight.

46. (canceled)

47. The process of claim 45, wherein the extragranular lubricant is magnesium stearate.

48. (canceled)

49. The process of claim 45, wherein the extragranular lubricant content is from about 1 wt % to about 2 wt %, based on the pharmaceutical dosage unit tablet core weight.

50.-52. (canceled)

53. The process of claim 18, wherein the divarasib freebase content is from about 1 wt % to about 44 wt %, based on the pharmaceutical dosage unit tablet core weight.

54. (canceled)

55. The process of claim 18, wherein the divarasib freebase content is about 40 wt %, based on the pharmaceutical dosage unit tablet core weight.

56. (canceled)

57. The process of claim 18, wherein divarasib is divarasib adipate at a content from about 1 wt % to about 60 wt %, based on the pharmaceutical dosage unit tablet core weight.

58.-60. (canceled)

61. The process of claim 57, wherein the divarasib adipate content is about 54 wt %, based on the pharmaceutical dosage unit tablet core weight.

62. (canceled)

63. (canceled)

64. The process of claim 18, wherein the pre-blend is compacted between at least two rotating rolls having a gap width of from about 1 mm to about 6 mm to form a ribbon, wherein the ribbon is milled and screened to form the granules.

65. (canceled)

66. (canceled)

67. The process of claim 18, wherein the bulk density of the granule blend is from about 0.4 g/mL to about 0.75 g/mL.
